(19) United States
(12) Patent Application Publication
(10) Pub. No.: US 2024/0201197 A1
(43) Pub. Date: Jun. 20, 2024

(54) Ultrasensitive Label-Free Profiling of Glycans Released from Single Cells

(71) Applicant: Northeastern University, Boston, MA (US)

(72) Inventors: Alexander IVANOV, Newton, MA (US); Anne-Lise MARIE, Brighton, MA (US); Yunfan GAO, Somerville, MA (US)

(21) Appl. No.: 18/542,532

(22) Filed: Dec. 15, 2023

Related U.S. Application Data
(60) Provisional application No. 63/432,953, filed on Dec. 15, 2022.

(51) Int. Cl. G01N 33/68; G01N 27/447

(52) U.S. Cl. CPC G01N 33/68; G01N 27/44743; G01N 27/44791; G01N 2333/98; G01N 2400/10; G01N 2560/00; G01N 2570/00

(57) ABSTRACT

An integrated platform is provided for direct and unbiased label-free native analysis of N-glycans from single cells and biological samples as small as 1 nL or less. An in-capillary sample processing method is coupled with high-sensitivity label-free capillary electrophoresis and mass spectrometry. Direct, label-free characterization and quantification of single-cell surface N-glycomes can be performed with the detection of up to 100 N-glycans per single cell and up to 400 N-glycans per nL of blood. N-glycome alterations can be detected at the single-cell level for diagnosis of medical conditions. The platform also preserves cell integrity and therefore can be used for spatial glycomic and multiomic profiling at the single cell level.

### CROSS REFERENCE TO RELATED APPLICATIONS

This application claims the priority of U.S. Provisional Application No. 63/432,953, filed 15 Dec. 2022 and entitled “Ultra-Sensitive Label-Free Deep Profiling of Glycans Released from Single-Cells and nL-Volumes of Amount-Limited Biomedically-Relevant Samples”, the whole of which is hereby incorporated by reference.

### STATEMENT REGARDING FEDERALLY SPONSORED RESEARCH OR DEVELOPMENT

This invention was made with government support under Grant Nos. 1R01CA218500 and 1R35GM136421 awarded by the National Institutes of Health. The government has certain rights in the invention.

### BACKGROUND

Glycans are assemblies of linear and branched monosaccharide chains that govern molecular interactions and, therefore, cell communication, signal transduction, pathogen recognition, and immune responses¹⁻³. In living mammalian cells, glycosylation (catalyzed by glycosyltransferases and glycosidases) produces a highly complex and vast repertoire of cellular glycans, with a colossal structural diversity¹. Mammalian cells are covered with a dense layer of glycans and the proteins and lipids they are attached to, termed the glycocalyx, which is involved in various vital cellular processes⁴⁻⁶. The type, size, structure, and charge of cell surface glycans may affect the biological properties of the cells and their susceptibility to potential viral infections^{2,4,7}. Mammalian glycoconjugates, located on the cell membrane and extra- or intracellular space, play crucial roles in physiological and pathological events^{2,4,5,7,8}. Alterations in the glycomic profiles of glycoproteins, e.g., overexpression of sialylated or core fucosylated glycans, or increased levels of complex-type branched glycans, may promote the acquisition of cellular features required for the malignant transformation of cells^{1,9-12}. Recent studies have also reported altered glycosylation patterns in patients with Alzheimer's disease^{13,14} Glycosylation abnormalities represent an overt source of potential biomarkers for the diagnostic, prognostic, and treatment monitoring of various human diseases, including autoimmune, congenital, oncological, and neurodegenerative pathologies^{5,7,12}. In the emerging field of glycomedicine, novel therapeutic drugs or vaccines targeting tumor-associated carbohydrate antigens, like Lewis antigens and polysialic acids, are also currently in clinical evaluation^{5,15,16}.

The analysis of single cells may result in the characterization of distinct cell subpopulations and rare cells, and in the differentiation of various cell states, which are often overlooked in bulk sample analyses^{17,18}. Single-cell analysis is also beneficial to certain applications (e.g., minimally invasive liquid biopsy/microbiopsy-based cancer diagnosis and monitoring), where the amounts of original biological material may be limited for downstream molecular profiling. Single-cell omics encompasses a broad spectrum of analytical techniques that rely on vastly different technological principles, including genomics, transcriptomics, metabolomics, lipidomics, proteomics, and glycomics^{17,20,21}. In contrast to single-cell genomics and transcriptomics, single-cell proteomics and glycomics remain in their infancy. The analysis of proteomes and glycomes at the single-cell level is limited by the minute amounts of starting biological material and the inability to directly amplify protein and glycan species. For single-cell proteomics, ultra-sensitive mass spectrometry (MS)-based analytical platforms are under development²²⁻²⁵. The Slavov group developed Single Cell ProtEomics by Mass Spectrometry (SCoPE-MS) techniques that rely on liquid chromatography (LC)-MS/MS methods with tandem mass tag (TMT)-labeling of peptides and a carrier channel for quantifying protein covariation across hundreds and potentially thousands of single cells^{26,27}. The Kelly^{28,29} and Mechtler³⁰ groups succeeded in developing reliable platforms for label-free single-cell proteomics using in-house or commercial nanoliter-liquid handlers. Gebreyesus et al. implemented a fully automated workflow combining microchips and MS for sample preparation and bottom-up analysis, which resulted in the detection of >1,500 protein groups from one single mammalian PC-9 cell³¹. Some of the present inventors have recently reported a capillary electrophoresis (CE)-MS method for top-down single-cell proteomic profiling³². This proof-of-concept approach allowed identification of >60 unique proteoforms in single HeLa cells³³.

Contrary to the rapidly growing field of single-cell proteomics, single-cell glycomics (SCG) has shown lagging progress. This may be explained by i) the substantially lowered amounts of biological material available (glycosylation may account for only ˜1-10% of the total mass of one human glycoprotein¹), and ii) the necessity to label the native glycans in the most commonly used positive electrospray ionization (ESI)-MS mode for increased ionization efficiency and detectability of released glycans, which obviously induces additional and substantial sample losses during the sample processing. Analytical platforms were implemented for total cellular glycome analysis of various mammalian cells, but these studies required large amounts of cells (˜10⁵-10⁷ cells)^{34,35}. To date, only a few analytical technologies have been developed for SCG. The Johnston group developed the SUrface-protein Glycan And RNA-seq (SUGAR-seq) method for the analysis of the transcriptome, extracellular epitopes, and N-linked glycosylation at the single-cell level, using biotinylated lectins and anti-biotin antibodies combined with multimodal RNA-seq technology³⁶. Oligonucleotide-labeled lectins were used by the Tateno group for sequencing-based glycomic profiling of single cells, which enabled the acquisition of 39 lectin-binding signals per single cell and provided an informative picture of cell surface glycosylation³⁷. Roan and co-workers implemented a cytometry time-of-flight-lectin (CyTOF-Lec) technique for the simultaneous detection and quantification of proteins and glycans on the surface of human cells³⁸. In this approach, lanthanide-conjugated lectins were combined with traditional CyTOF mass cytometry using lanthanide-conjugated antibodies to specifically bind glycans and proteins and quantify them (through the lanthanide metal quantification) using inductively-coupled-plasma-MS (ICP-MS). The developed technique showed that CD4+ T cell surface glycosylation could influence the susceptibility of CD4+ T cells to viral infection. Nevertheless, the above-described approaches involve tedious, expensive, and time-consuming analytical workflows with sophisticated instrumentation and, most importantly, do not allow the direct analysis, quantitation, and accurate structural characterization of the glycans. Furthermore, some cell surface glycans may not interact with the lectins selected in the developed methodologies. These last few years, computational modeling software tools to predict the glycome at the single-cell level were also developed, based, for example, on single-cell RNA-seq transcriptomics data 39. Yet, so far, methods enabling the direct analysis, characterization, and quantitation of cell glycomes at the single-cell level have not been reported.

### SUMMARY

The present technology provides an integrated platform coupling online in-capillary sample processing with high-sensitivity label-free capillary electrophoresis-mass spectrometry for N-glycan profiling of single mammalian cells and nL-volumes of amount-limited biomedically-relevant samples. Direct and unbiased characterization and quantification of single-cell surface N-glycomes were demonstrated for HeLa and U87 cells, with the detection of up to 100 N-glycans per single cell. N-glycome alterations were unequivocally detected at the single-cell level in HeLa and U87 cells stimulated with lipopolysaccharide. The developed method was also applied to the profiling of ng-level amounts of blood-derived protein, extracellular vesicles, and total plasma isolates, resulting in over 170, 220, and 370 quantitated N-glycans, respectively.

An aspect of the present technology is a method of glycan analysis including the following steps: (a) providing an open tube capillary electrophoresis instrument whose output provides an electrospray or other ionization-based input for a mass spectrometer, a glycan release agent solution disposed within a capillary tube of the open tube capillary electrophoresis instrument, and a sample comprising a glycoprotein or glycolipid in an aqueous medium; (b) introducing the sample into an inlet of a capillary tube of the open tube capillary electrophoresis instrument or the channel of a microfluidic capillary electrophoresis instrument, whereby the glycoprotein or glycolipid contacts the glycan release agent solution; (c) allowing the glycan release agent to release one or more glycan moieties from the glycoprotein or glycolipid without modification of glycan structure or composition; (d) separating the released glycan moieties within the capillary tube or channel using the open tube capillary electrophoresis instrument or the microfluidic capillary electrophoresis instrument based on charge and hydrodynamic volume of the released glycan moieties to form a plurality of separated glycan moieties within the capillary tube; (e) injecting the separated glycan moieties from an outlet of the capillary tube or channel into the mass spectrometer, whereby the separated glycan moieties are ionized and fragmented to form a plurality of charged glycan fragments; (f) separating and detecting the charged glycan fragments based on mass-to-charge ratio using the mass spectrometer; and (g) analyzing the separated and detected charged glycan fragments, whereby one or more structural characteristics of said plurality of glycan moieties are determined. The glycan release agent can be a single enzyme, such as PNGase F, or a combination of enzymes, or a chemical reagent. In the present technology, glycans are released from glycoproteins or glycolipids, preferably as an intact unit with its native structure preserved and without chemical derivatization or labeling, through an enzymatic reaction carried out in the capillary tube of a capillary electrophoresis instrument. The method allows separation, identification, and quantification of glycans in a single run and avoids sample manipulations that lead to sample losses or alteration.

The present technology can also be summarized in the following list of features.

1. A method of glycan analysis, the method comprising the steps of:
(a) providing an open tube capillary electrophoresis instrument whose output provides an electrospray input for a mass spectrometer, a glycan release agent solution disposed within a capillary tube of the open tube capillary electrophoresis instrument, and a sample comprising a glycoprotein in an aqueous medium;
(b) introducing the sample into an inlet of a capillary tube of the open tube capillary electrophoresis instrument, whereby the glycoprotein contacts the glycan release agent solution;
(c) allowing the glycan release agent to release one or more glycan moieties from the glycoprotein without modification of glycan structure or composition;
(d) separating the released glycan moieties within the capillary tube using the open tube capillary electrophoresis instrument based on charge and hydrodynamic volume of the released glycan moieties to form a plurality of separated glycan moieties within the capillary tube;
(e) injecting the separated glycan moieties from an outlet of the capillary tube into the mass spectrometer, whereby the separated glycan moieties are ionized and fragmented to form a plurality of charged glycan fragments;
(f) separating and detecting the charged glycan fragments based on mass-to-charge ratio using the mass spectrometer; and
(g) analyzing the separated and detected charged glycan fragments, whereby one or more structural characteristics of said plurality of glycan moieties are determined.
   2. The method of feature 1, wherein the glycan release agent is PNGase F, and the released glycan moieties are N-glycans.
   3. The method of feature 1, wherein the one or more glycans are released, separated, fragmented, and analyzed from glycoproteins of 1 to about 20 single cells present in the sample.
   4. The method of feature 3, wherein one or more glycans are released, separated, fragmented, and analyzed from glycoproteins of a single cell.
   5. The method of any of the preceding features, wherein the analyzing of step (g) results in complete structure elucidation of at least one glycan moiety.
   6. The method of feature 5, wherein the complete structure elucidation comprises identification of all monosaccharides of the glycan moiety and glycan derivative moieties and identification of glycosidic linkages of the glycan moiety.
   7. The method of feature 5 or 6, wherein the complete structure elucidation identifies an intact native structure of the glycan moiety.
   8. The method of feature 6, wherein the structural elucidation comprises the identification of glycan moieties containing from 1 to about 20 sialic acid residues.
   9. The method of feature 6, wherein the structural elucidation comprises the identification of glycan moieties containing from 1 to about 10 fucose residues.
   10. The method of any of the preceding features, wherein the analyzing of step (g) comprises using output of peak areas and intensities from the mass spectrometer to enable quantitative analysis of released glycans.
   11. The method of any of the preceding features wherein, in step (d), isomers of glycan moieties are separated within the open tube capillary electrophoresis instrument in a single run.
   12. The method of any of the preceding features, wherein the sample comprises one or more single cells and the method preserves integrity of analyzed single cells.
   13. The method of any of the preceding features, wherein the method enables identification and quantification of intact and native glycan moieties.
   14. The method of any of the preceding features, wherein the method does not comprise labeling or derivatizing any glycan moiety.
   15. The method of any of the preceding features, wherein the method does not comprise use of any other endoglycosidase or exoglycosidase.
   16. The method of any of the preceding features, wherein glycans from at least 10, at least 20, at least 30, at least 40, or at least 50 different glycoproteins are released, separated, fragmented, and analyzed in a single run.
   17. The method of any of the preceding features, wherein the sample has a volume of less than 1 μL.
   18. The method of feature 17, wherein the sample has a volume of less than 1 nL.
   19. The method of any of the preceding features, wherein the sample is obtained from blood, plasma, a bodily fluid, a biopsy sample, a cell suspension, a subcellular fraction, or a cell culture, or an extract or fraction of any of the foregoing.
   19a. The method of feature 19, wherein the blood, plasma, bodily fluid, biopsy sample, cell suspension, subcellular fraction, cell culture, or extract or fraction thereof was obtained from a source previously contacted with a chemical or biological therapeutic agent.
   20. The method of any of the preceding features, wherein the sample is cell-free.
   21. The method of any of the preceding features, wherein the sample is not subjected to any purification, homogenization, chromatography, centrifugation, or fractionation prior to use in the method.
   22. The method of any of features 1-20, wherein the sample is subjected to purification, homogenization, chromatography, centrifugation, or fractionation prior to use in the method.
   23. The method of any of the preceding features, wherein the separated glycan moieties are subjected to electrospray ionization when injected into the mass spectrometer in step (e).
   24. The method of any of the preceding features, wherein capillary electrophoresis/tandem mass spectrometry (CE/MS/MS) is used to perform steps (b) through (f).
   25. The method of any of the preceding features, wherein hydrodynamic pressure, electrospray, or electrokinetic injection is used to introduce the sample into the inlet of the capillary tube in step (b).
   26. The method of any of the preceding features, wherein the method is capable of full structure elucidation of a glycan present in a sample in amounts over a range of at least 4 or at least 5 orders of magnitude.
   27. The method of any of the preceding features, wherein the method is used to aid in performing spatial glycomic profiling of single cells or non-cellular sub-nanogram samples.
   28. The method of any of the preceding features, wherein the method is used to aid in performing spatial multiomic profiling of single cells or non-cellular sub-microgran or sub-nanogram quantity samples.
   29. The method of feature 28, wherein the multiomic profiling comprises glycomic profiling and one or more of proteomic, genomic, transcriptomic, and metabolomic profiling.
   30. The method of any of the preceding features, wherein the method is used to aid in diagnosis and/or treatment of a disease or medical condition, and wherein the sample is obtained from a subject suspected of having the disease or medical condition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show a CE-MS-based experimental workflow for N-glycan profiling of single mammalian cells and blood-derived isolates. FIG. 1A presents a schematic representation of the analytical platform. Individual mammalian cells are injected manually using the height difference between the inlet and outlet ends of the CE capillary. The single-cell plug is sandwiched between two plugs (1 nL each) of a PNGase F digestion solution. After 30 min (blood-derived isolates) or 1 h (mammalian cells) incubation with PNGase F, the CE and MS electrospray voltages are triggered for label-free CE-MS analysis of released N-glycans. FIG. 1B shows a representative image of a HeLa cell suspension droplet used for single-cell loading. FIG. 1C shows an averlay of bright-field and fluorescence images showing one single HeLa (top view) and three HeLa (bottom view) cells loaded into the CE capillary. FIG. 1D shows comparative bright-field (left) and fluorescence (right) images displaying cell integrity before (T0) and after (T1) the in-capillary deglycosylation step with PNGase F.

FIGS. 2A-2K show CE-MS-based N-glycan profiling of human blood-derived isolates. FIG. 2A shows the number of N-glycans (n=3) identified in four analyzed blood-derived isolates (human serum IgM, human serum IgG, total plasma, and EV isolate). FIGS. 2B-2H) show fractional distributions of fucosylated N-glycans detected in IgM (2B), IgG (2D), total plasma (2F), and EVs (2H). FIG. 2C-2I show fractional distributions of sialylated N-glycans detected in IgM (C), IgG (E), total plasma (G), and EVs (I). FIG. 2J shows a Euclidean distance-based hierarchical clustering of quantitative glycomic profiles of IgM, IgG, total plasma, and EVs, using injected amounts of 5 ng of IgM, 5 ng of IgG, 500 pL of plasma, and 50 nL of EV isolate (corresponding to −150 nL of plasma), respectively. Red, yellow, and light blue colors correspond to high, medium, and low relative abundances based on the normalized N-glycan signal intensities. N-glycans that are not detected in the blood-derived samples are highlighted in dark blue. FIG. 2K shows label-free CE-MS-based profiling of N-glycans released from human blood isolates (IgM, IgG, total plasma, and EVs). The Venn diagram shows the overlap of N-glycans detected in the four analyzed blood-derived samples.

FIGS. 3A-3L show CE-MS-based N-glycan profiling of HeLa and U87 mammalian cells. FIGS. 3A-3F show representative ion density maps acquired in label-free CE-MS analyses of N-glycans released from (3A) one HeLa cell, (3B) five HeLa cells, (C3) ˜ten HeLa cells, (3D) one U87 cell, (3E) water blank sample, and (3F) cell suspension medium (a normalized intensity level of 3.9×10⁴ was selected for each density map). FIG. 3G shows the number of N-glycans (n=5) identified in the analyzed mammalian cell and control samples. FIGS. 3H-3K show Venn diagrams illustrating the overlap of N-glycans identified in five repetitive analyses acquired with the injection of one (3H), five (3I), and ˜ten (3J) HeLa cells, and one U87 cell (3K). FIG. 3L shows a Venn diagram illustrating the overlap of N-glycans identified in single HeLa and single U87 cells, based on the total number of N-glycans identified in HeLa and U87 single cells (see FIGS. 3H and 3K, respectively).

FIGS. 4A-4K present the fractional distributions of fucosylated and sialylated N-glycans detected in HeLa and U87 mammalian cells. FIGS. 4A-4G show fractional distributions of fucosylated N-glycans detected in one, five, and ˜ten HeLa cells (4A), HeLa and U87 single cells (4C), LPS-treated and untreated single HeLa cells (4E), and LPS-treated and untreated single U87 cells (4G). FIGS. 4B-4H show the fractional distributions of sialylated N-glycans detected in one, five, and ˜ten HeLa cells (4B), HeLa and U87 single cells (4D), LPS-treated and untreated single HeLa cells (4F), and LPS-treated and untreated single U87 cells (4H). FIG. 4I shows normalized abundances of eleven representative N-glycans detected in one, five, and ˜ten HeLa cells, and one U87 cell. Glycan symbols: blue square, GlcNAc; red triangle, Fuc; green circle, Man; yellow circle, Gal; purple diamond, Neu5Ac. FIG. 4J shows the number of N-glycans (n=5) identified in single HeLa and single U87 cells before and after LPS stimulation. FIG. 4K shows a Venn diagram illustrating the overlap of N-glycans identified in single HeLa and single U87 cells with and without LPS treatment, based on the total number of N-glycans identified in the respective analyzed samples.

FIGS. 5A-5D show CE-MS²-based structural characterization of representative HeLa and U87 cell-derived N-glycans. FIGS. 5A-5D show characteristic CE-MS² spectra of (5A) the fucosylated disialylated glycan FA2G2S2, (5B) the trisialylated glycan A3G3S3, (5C) the tetrasialylated glycan A3G3S4, and (5D) the pentasialylated glycan A3G3S5, selecting the molecular ions at m/z 1,183.42, 958.66, 1,055.69, and 1,152.73 as precursor ions, respectively. Fragment ions are annotated based on the Domon and Costello nomenclature. Blue square, GlcNAc; red triangle, Fuc; green circle, Man; yellow circle, Gal; purple diamond, Neu5Ac. Symbol Z indicates cross-ring fragmentation. Only the most intense/relevant fragments are annotated in the shown spectra.

FIGS. 6A-6F show differential qualitative and quantitative N-glycan profiling of single HeLa and single U87 mammalian cells. FIGS. 6A-6C show Euclidean distance-based hierarchical clustering of N-glycans detected in (6A) HeLa and U87 cells, and (6B) LPS-treated and untreated HeLa and U87 cells, based on five repetitive analyses of single HeLa and single U87 cells (labeled from 1 to 5). Red, yellow, and light blue colors correspond to high, medium, and low relative abundances based on the normalized N-glycan signal intensities. N-glycans that are not detected in the cell samples are highlighted in dark blue. FIGS. 6B-6D show principal component analysis (PCA) of N-glycans detected in HeLa and U87 cells (6B), and PCA of LPS-treated and untreated HeLa and U87 cells (6D). FIGS. 6E-6F show total abundances of fucosylated and nonfucosylated (6E) and sialylated and nonsialylated (6F) N-glycans detected in HeLa and U87 single cells, before and after LPS treatment, based on the normalized N-glycan signal intensities.

FIGS. 7A-7P show the relative abundance levels of the N-glycans detected in four analyzed blood-derived samples, IgM, IgG, plasma, and extracellular vesicles (EVs).

FIGS. 8A-8F show structural characterization of 53 N-glycans in HeLa cells.

FIGS. 9A-9C show characteristic CE-MS² spectra of high mannose-type N-glycans detected in HeLa cells. The MS²-based structural characterization of Man6 (9A) and Man7 (9B) was performed by selecting the [M−1H]¹⁻ molecular ions at m/z 1395.47 and 1579.53, respectively. The MS²-based structural characterization of Man13 (9C) was performed by selecting the [M−2H]²⁻ molecular ion at m/z 1275.42.

FIGS. 10A-10F show qualitative and quantitative comparison of single-cell N-glycomes before and after the stimulation of the cells with LPS. Venn diagrams illustrating the overlap of N-glycans identified in single HeLa cells before and after LPS treatment (10A), and identified in single U87 cells before and after LPS treatment (10B), based on five repetitive analyses for each cell line. Fractional abundances of fucosylated N-glycans detected in single HeLa (10C) and single U87 (10E) cells before and after LPS treatment. Fractional abundances of sialylated N-glycans detected in single HeLa (10D) and single U87 (10F) cells before and after LPS treatment.

### DETAILED DESCRIPTION

The inventors developed an integrative platform coupling on-line in-capillary sample processing with high-sensitivity label-free capillary electrophoresis-mass spectrometry (CE-MS) for N-glycan profiling of single mammalian cells and limited amounts of biomedically-relevant specimens (e.g., blood-derived isolates). The biological samples (e.g., individual cells, serum proteins, total plasma, and extracellular vesicles (EVs) were tested) are injected into the CE capillary by hydrodynamic pressure. Then, underivatized and native N-glycans are released within the capillary with a digestion solution of PNGase F enzyme. Finally, the released N-glycans are analyzed by high-sensitivity CE-MS in their non-labeled state without any further sample preparation. The developed technique allows, for the first time, direct and unbiased characterization and quantification of single mammalian cell N-glycomes.

To date, only a few analytical technologies, based on carbohydrate-binding lectins, have been developed for single-cell glycomics. These technologies, which require sophisticated instrumentation, are very expensive and time-consuming, and, most importantly, result in undirect and biased profiling of cell-surface glycans (i.e., the glycans are not released from the cell surface for their direct detection and quantification, and the glycan detectability is dependent on lectin binding affinity). Furthermore, some cell surface glycans may not interact with the lectins selected in the developed methodologies. The present technology offers a vastly simplified in-capillary sample preparation approach coupled on-line with label-free CE-MS. With this method, the glycans are analyzed by CE-MS in their native non-labeled state, and the substantial sample losses associated with sample handling and transfer steps in the off-line approach are eliminated. This present innovative workflow allows straightforward and effective glycan profiling of small populations of cells (1-10 cells) as well as nL down to pL levels of biomedically-relevant samples. CE-MS analysis of N-glycans in their native non-labeled state enables the preservation of the integrity and endogenous structural features of glycans, especially fucosylation and sialylation. In contrast, the most commonly used glycan labeling approaches can substantially alter the biological sample and result in partial disintegration of glycans during the process of labeling and sample preparation. Besides, the label-free CE-MS method, the present technology provides accurate and unambiguous structural characterization of the detected glycans, and provides crucial information on glycan antenna-branching and glycosidic linkages, without using selective enzymatic digestions (a tedious and time-consuming approach commonly used in glycomic analysis, which may leave some ambiguity in the assignment of glycosidic linkages).

The mild conditions used for N-glycan release allowed preservation of cell membrane integrity and specifically liberated cell surface N-glycans. N-glycans were analyzed in their native underivatized state to preserve their endogenous glycan features and eliminate the drawbacks associated with any labeling procedures, including incomplete derivatization, side-products, sample losses during cleanup steps, and high levels of defucosylation/desialylation during sample preparation and MS analysis. For glycan analysis of intact mammalian cells (1-10 cells), a manual hydrodynamic cell loading procedure³² was further optimized, not only to increase the robustness and throughput of cell loading, but also to improve the detectability and separation of the released N-glycans during CE-MS analysis. The vastly simplified in-capillary sample preparation approach coupled online with CE-MS eliminated sample losses associated with sample handling and transfer steps in the offline approach and allowed us to analyze sub-ng-levels of model proteins and pL-levels of total plasma, as well as single mammalian cells. Such an approach enabling direct analysis and quantification of N-glycans derived from one single cell has not been reported previously. In addition, biochemical stimulation of mammalian cells induced significant qualitative and quantitative changes in cell glycosylation profiles, which confirmed the potential of the method to detect cell glycome alterations in biological and biomedical applications at the single-cell level.

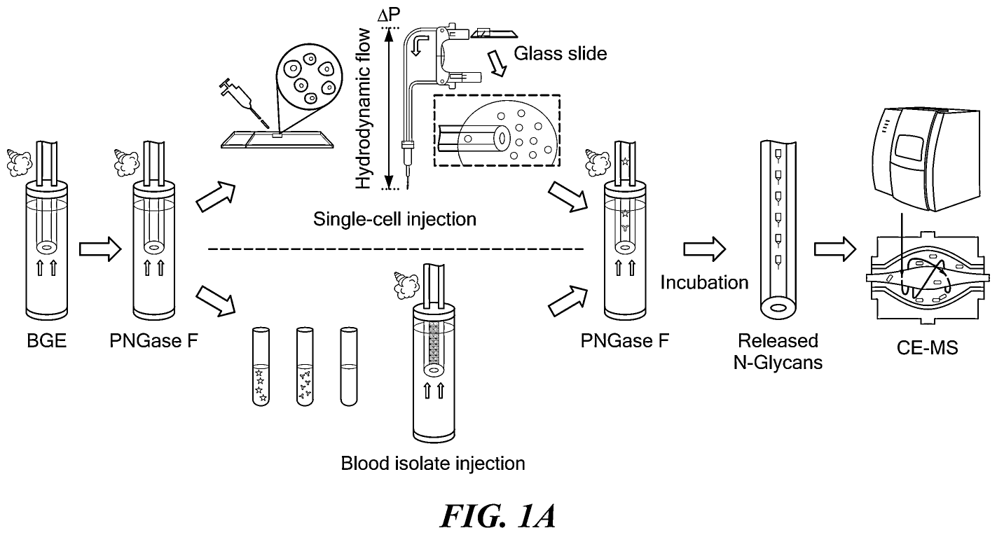

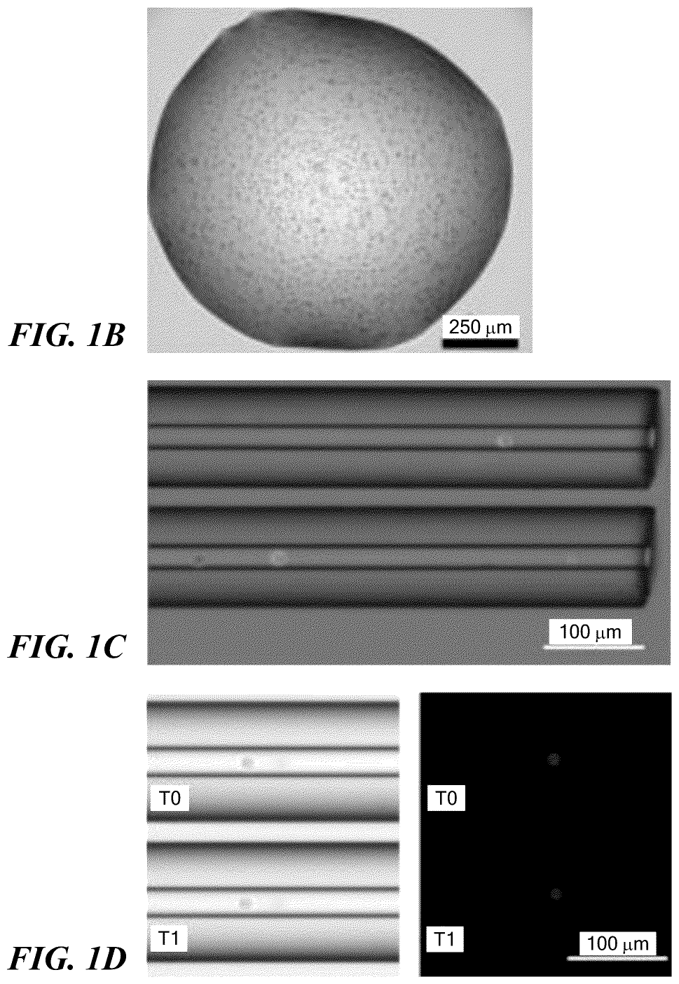

FIG. 1A depicts s method of in-capillary sample processing, N-glycan release, and glycan characterization according to the present technology. The method can be used for N-glycan profiling of single cells as well as for limited amounts of blood-derived isolates or other biological samples. The injected samples were sandwiched between two plugs of PNGase F and incubated inside the capillary. After the digestion step, the CE and ESI-MS voltages were triggered for online CE-MS analysis of the released N-glycans in their native non-labeled state.

### Glycan Release

The present methods utilize in-line, in-instrument sample processing, separation, and analysis, all in a single continuous process. A key aspect is the release of glycan moieties from glycoproteins and glycolipids at the outset of the method, via one or more enzyme reactions carried out within the capillary tube or microfluidic channel of a capillary electrophoresis instrument. In a preferred embodiment, a single reaction is carried out in a liquid “plug” in the capillary containing the sample or a portion thereof and one or more glycan release agents. A glycan release agent is capable of releasing glycan moieties, preferably in there entirety and in their native state, as found in a cell or in the biological source of the glycoprotein or glycolipid. Also preferred is that a single glycan release agent is used that releases complete glycan moieties in a single step. However, also suitable can be two or more enzymes that work together to release portions of glycans either in a single step (i.e., performed in a single plug in the capillary, or in a series of sequential steps performed in a sequential series of plugs, each containing a different enzyme (e.g., endoglycosidases and/or exoglycosidases).

Any suitable glycan release agent can be used in the present technology. A preferred glycan release agent for N-glycans is PNGase F, which cleaves off N-glycans in their entirety and in their native structure from N-linked glycoproteins. For O-glycans, released from O-linked glycoproteins, monosaccharides can be sequentially released by a series of exoglycosidases until only the Gal-β(1→3)-GalNAc core remains. O-Glycosidase can then remove the intact core structure with no modification of the serine or threonine residues. Chemical reagents that release glycan moieties are known and also can be used. See Song, et al., 2016, Nat. Methods 13(6):528-534.

### N-Glycome Profiling of Blood-Derived Isolates

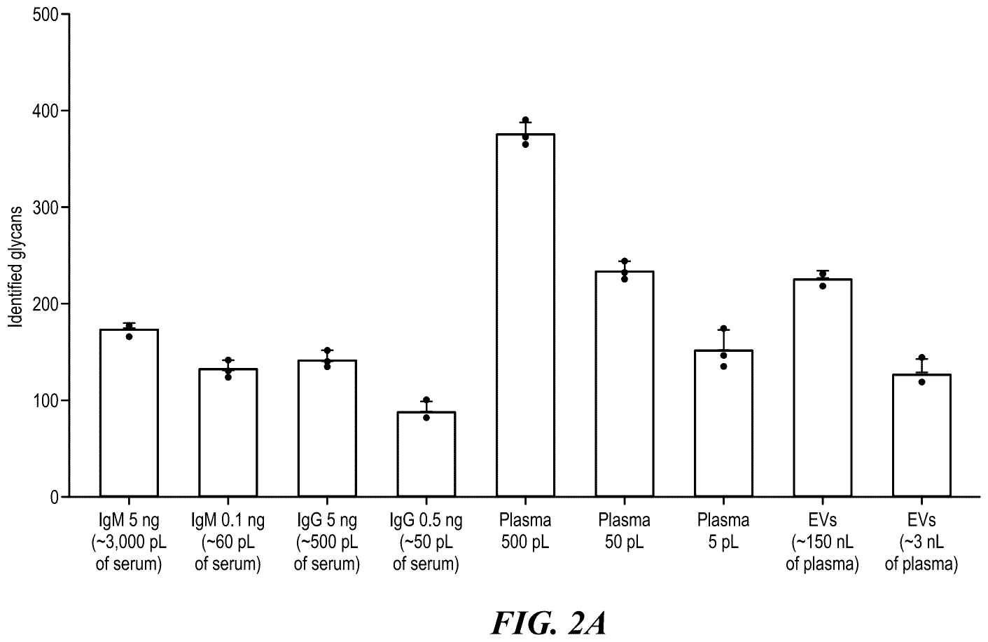

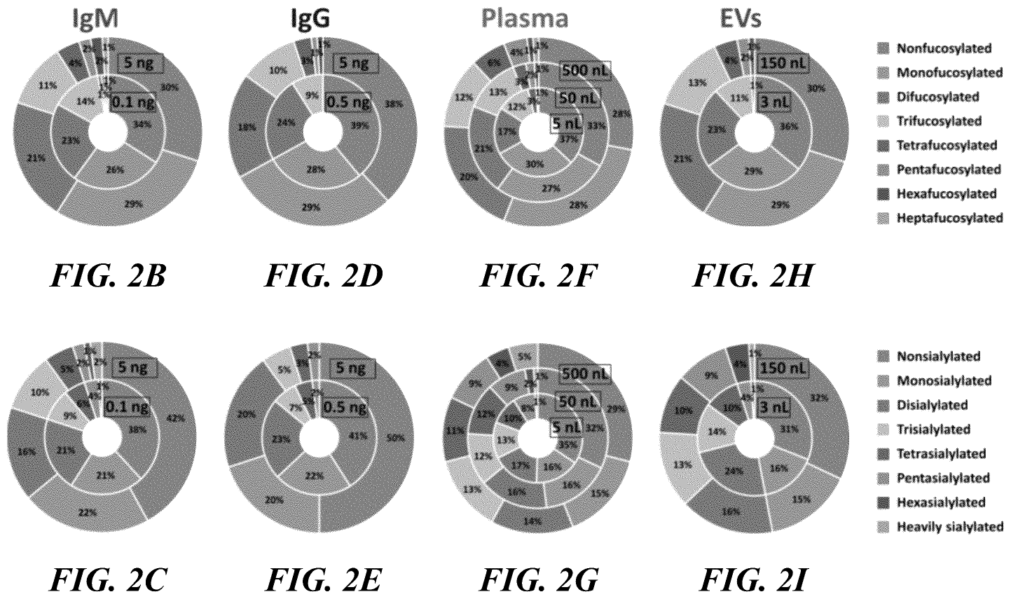

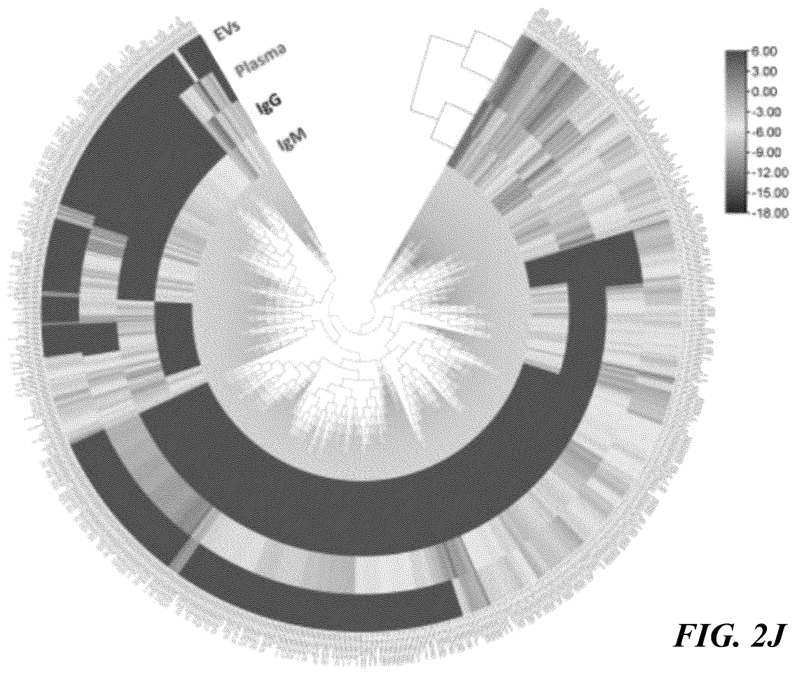

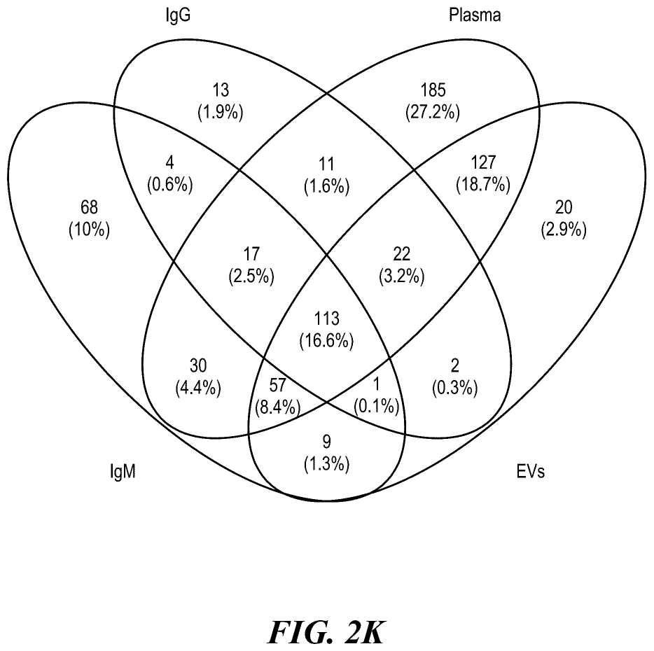

N-glycan profiling of human serum IgM. Human immunoglobulin M (IgM), a heavily glycosylated multimeric protein (Mrₜₕ 970 kDa and 1,080 kDa for pentameric and hexameric forms, respectively), was selected to develop and optimize the in-capillary sample processing method for N-glycan release coupled online to label-free CE-MS analysis. N-glycans account for ˜10% of the total mass of IgM 4, and the serum level of IgM is in the range 0.4-2.5 mg/mL^{45,46}. CE-MS analysis of IgM-derived in-capillary released N-glycans resulted in the identification of 173±6 (n=3) non-redundant N-glycan compositions in human serum IgM isolate for injected amounts of 5 ng (i.e., 5 fmol) of protein, corresponding to ˜500 pg of N-glycans and equivalent to the amount of IgM isolated from ˜3 nL of human serum (FIG. 2A). Interestingly, the injection of sample amounts as small as 0.1 ng (i.e., 100 amol) of IgM, corresponding to ˜10 pg of N-glycans and equivalent to ˜60 pL of human serum, resulted in the detection and identification of 132±9 (n=3) N-glycans (FIG. 2A). Considering the current developments in single-cell analysis, it is worth noting that these minute amounts of proteins and glycans (˜100 pg and ˜10 pg, respectively) are equivalent to the protein and glycan content of one single mammalian cell²⁰. Using the present label-free CE-MS-based workflow, the number of identified N-glycans was increased ˜7-fold, compared to previously reported studies focused on N-glycan profiling of human serum IgM^{47,48}. FIGS. 2B-2C displays the fractional distributions of fucosylated and sialylated N-glycans identified in IgM, using 0.1 ng and 5 ng injected amounts of IgM. While highly fucosylated (up to 6 fucose residues) and highly sialylated (up to 6 sialic acid (SiA) residues) N-glycans were detected in the CE-MS analyses performed with either 5 ng or 0.1 ng of IgM, heptafucosylated and heavily sialylated (7-11 SiA residues) N-glycans were detected only with 5 ng of IgM injected amounts, indicating the very low abundances of these uncommon glycans.

The optimized workflow described above was well adapted to the analysis of 5 ng and sub-ng amounts of serum IgM. Using these tiny sample amounts and intensive rinsing steps between runs, no significant carryover derived from the analysis of preceding IgM samples was observed, based on the analysis of the water blank control sample. The injection of larger amounts of protein (e.g., 25-100 ng), which could potentially increase the glycan coverage of IgM, would require the re-optimization of several parameters, including the glycosidase: protein substrate ratio, the incubation time, and the rinsing steps between runs to efficiently clean the capillary. This scale-up workflow would obviously increase the sample processing and total analysis times. Since the goal of our study was to develop an effective and quick CE-MS-based workflow applicable to single-cell analysis, It was estimated that glycan amounts released from the digestion of 0.1 to 5 ng of model glycoprotein within the CE capillary should reflect well the amounts of glycans released from one to ten mammalian cells.

N-glycan profiling of human serum IgG. The newly developed N-glycan profiling workflow was tested using CE-MS analysis of human IgG (Mrₜₕ 150 kDa), a class of immunoglobulin less glycosylated than IgM. N-glycans account for ˜2% of the total mass of IgG, and the human serum level of IgG is in the range 7-16 mg/mL^{45,46}. The developed CE-MS method allowed identification of 142±9 (n=3) non-redundant N-glycan compositions in human serum IgG isolate for injected amounts of 5 ng (i.e., 33 fmol) of protein, corresponding to ˜100 pg of N-glycans and equivalent to the amount of IgG isolated from −500 pL of serum (FIG. 2A). Compared to previous studies reporting N-glycan profiling of IgG^{40,41}, the injected amounts of IgG were decreased ˜5-fold. With such low injected amounts, the number of identified N-glycans still largely exceeded (˜6-fold) the number of N-glycans reported in human serum IgG by others^{49,50}. As observed for IgM, the injection of protein amounts larger than 5 ng of IgG, in order to increase the glycan coverage of IgG, would require a workflow re-optimization. The inventors deemed it more relevant to decrease the injected protein amount to mimic the amount of glycans released from one single cell. The injection of ˜0.5 ng (i.e., 3 fmol) of IgG resulted in the identification of 88±10 (n=3) N-glycans (FIG. 2A). These scarce amounts of IgG correspond to only ˜10 pg of N-glycans and isolates from −50 pL of serum. FIGS. 2D-2E display the fractional distributions of fucosylated and sialylated N-glycans identified in IgG. In previous work⁴⁰, it was shown that hexa- and heavily sialylated N-glycans could not be detected in CE-MS analysis of non-labeled IgG-derived N-glycans using injected amounts equivalent to ˜25 ng of IgG. Glycans with a degree of sialylation ≥6 were not detected in such low 0.5-5 ng IgG sample amounts using the present workflow, since even larger injected amounts of serum IgG also did not allow their detection previously.

N-glycan profiling of total human plasma. The above described method allowed analysis of sub-nL volumes of total human plasma isolate, and enabled direct online N-glycan profiling of plasma volumes as small as 5 pL, which was not reported before. Data processing of CE-MS analyses resulted in the identification of 375±12, 234±10, and 152±21 (n=3) non-redundant N-glycan compositions in whole blood plasma for injected amounts of 500 pL, 50 pL, and 5 pL of plasma (i.e., ˜1,500, 150, and 15 pL of human blood), respectively (FIG. 2A). In previous work 4° 210±12, and 62±31 N-glycans were identified in whole plasma for injected amounts equivalent to ˜160 pL and ˜80 pL of plasma, respectively, using label-free CE-MS analysis of plasma-derived glycans released offline. The present results, therefore, demonstrate the superior performance of the newly developed in-capillary sample processing-based workflow for straightforward and unbiased N-glycome analysis of minute amounts of physiological fluids. As shown in FIGS. 2F-2G, hexa- and heptafucosylated, and heavily sialylated (i.e., ≥7 SiA residues) N-glycans were not detected using ˜5 pL of plasma injected volumes. These highly fucosylated and sialylated N-glycans were only detected using larger plasma volumes. Glycans containing up to 13-14 SiA residues were detected with the injection of 50 and 500 pL of plasma.

N-glycan profiling of blood-derived extracellular vesicles. The developed in-capillary workflow was applied to the analysis of N-glycans released from human plasma-derived extracellular vesicles (EVs), another attractive source of disease biomarkers^{51,52}. Experiments were carried out with the injection of a purified EV isolate, containing ˜1×10⁴ EV particles/nL (see Methods section). CE-MS analysis resulted in the detection and identification of 127±14 and 226±7 N-glycans in the total EV isolate, using 1 nL and 50 nL of EV isolate injection volumes, respectively (containing approximately 1×10⁴ EVs and 5×10⁵ EVs, respectively) (FIG. 2A). These injected amounts are equivalent to the EV content of ˜3 nL and ˜150 nL of plasma, respectively. FIGS. 2H-2I display the fractional distributions of fucosylated N-glycans detected in the total EV isolate using injection volumes of 1 nL and 50 nL of the EV isolate (i.e., ˜3 and ˜150 nL of plasma equivalents). Interestingly, the injection of volumes as small as 1 nL of EV isolate resulted in the detection of tetrafucosylated and hexasialylated N-glycans. The injection of larger volumes of EV isolate (i.e., 50 nL) allowed increasing the coverage of fucosylated glycans with the detection of penta- and hexafucosylated N-glycans, which were not detected using 1 nL of EV isolate. The injection of 50 nL of EV isolate also resulted in the detection of heavily sialylated N-glycans (up to 14 SiA residues), undetectable using 1 nL volumes of EV isolate. Compared to a previous study 4, a similar coverage of EV-derived N-glycans was achieved using the newly developed label-free CE-MS technique, based on the number and types (i.e., degrees of fucosylation and sialylation) of identified N-glycans for similar injected amounts. Finally, the present CE-MS-based workflow allowed further expansion of the catalog of blood-derived EV N-glycans, compared to other studies reported for N-glycome profiling of biofluid-derived EVs^{41,53}, using injected amounts as low as ˜150 nL of plasma (i.e., ˜400 nL of blood).

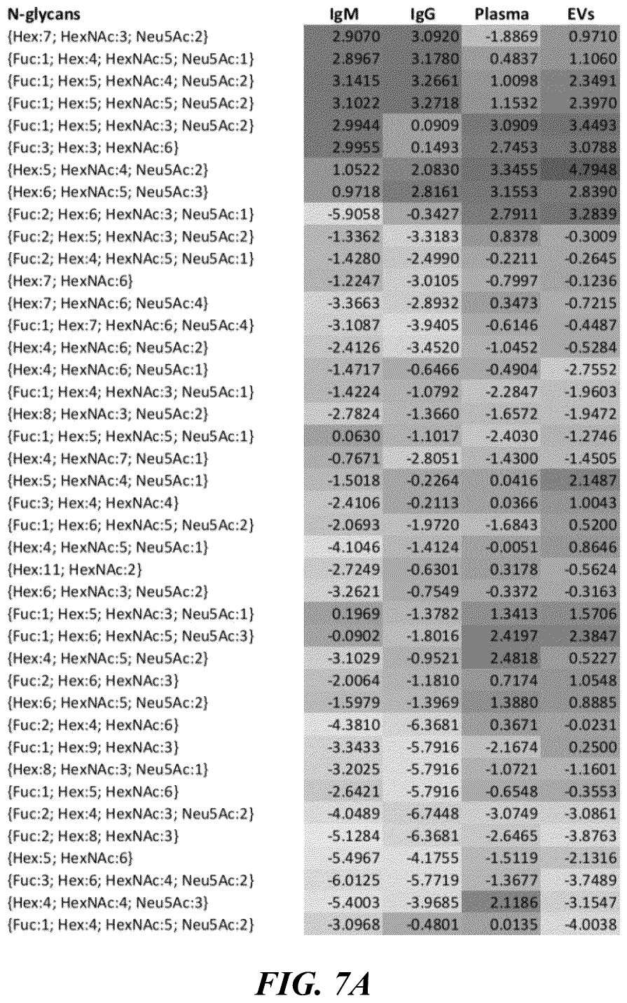

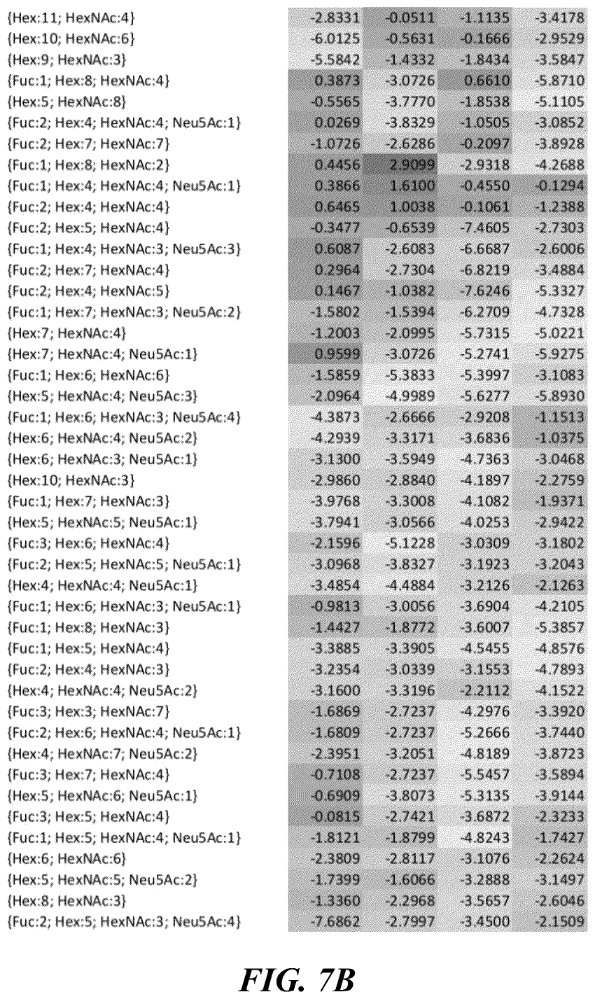

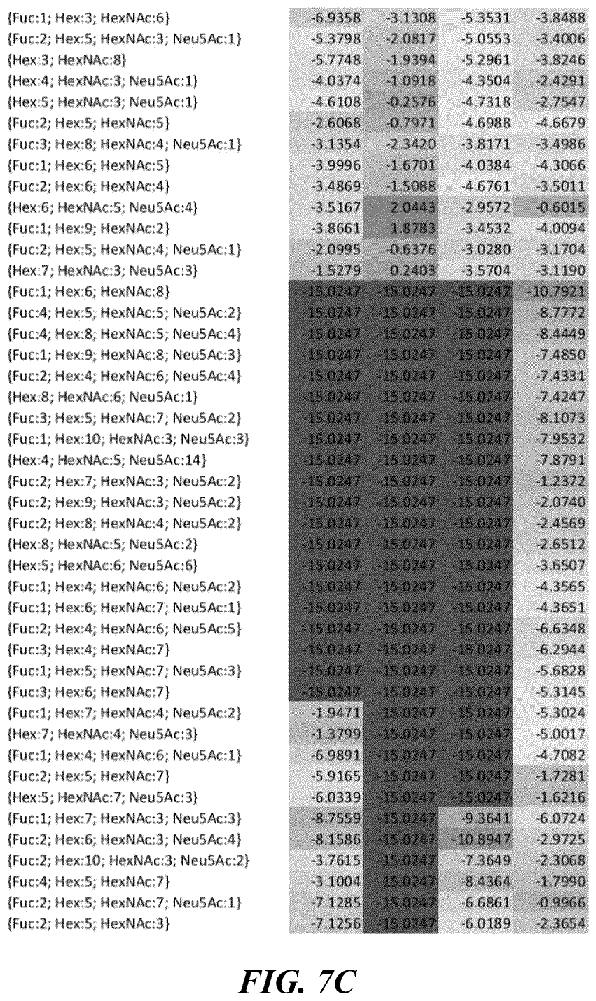

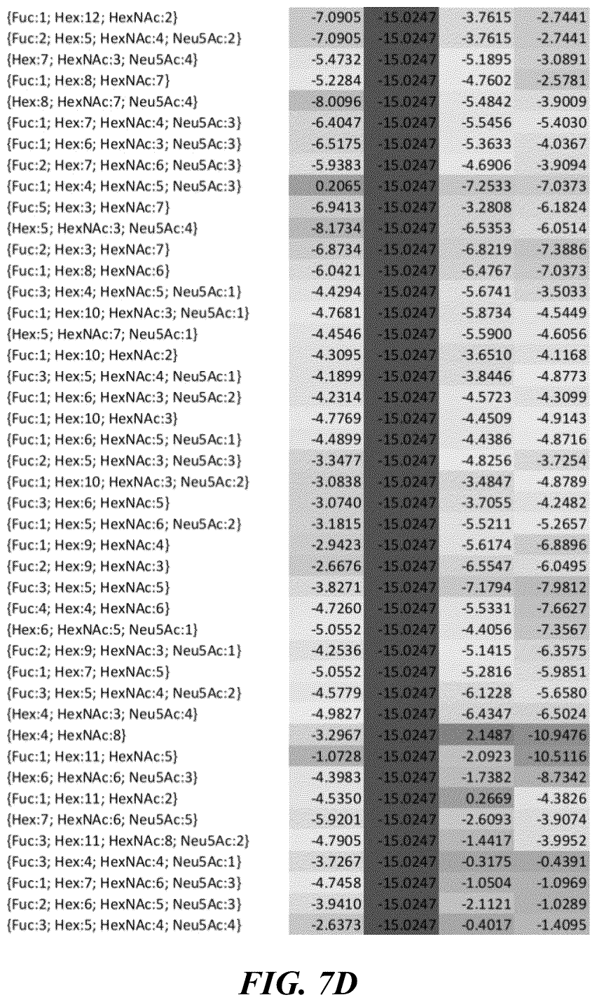

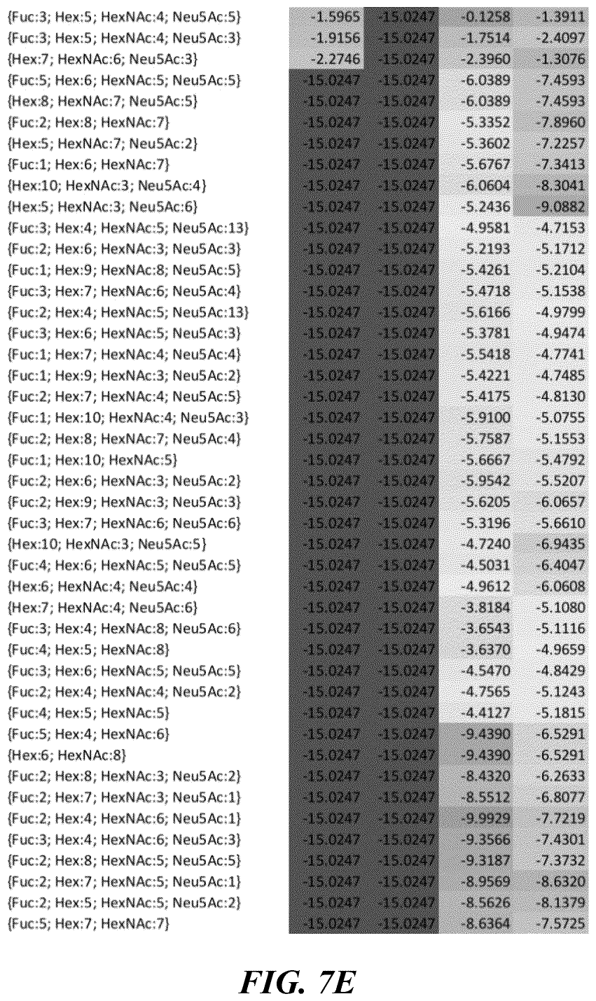

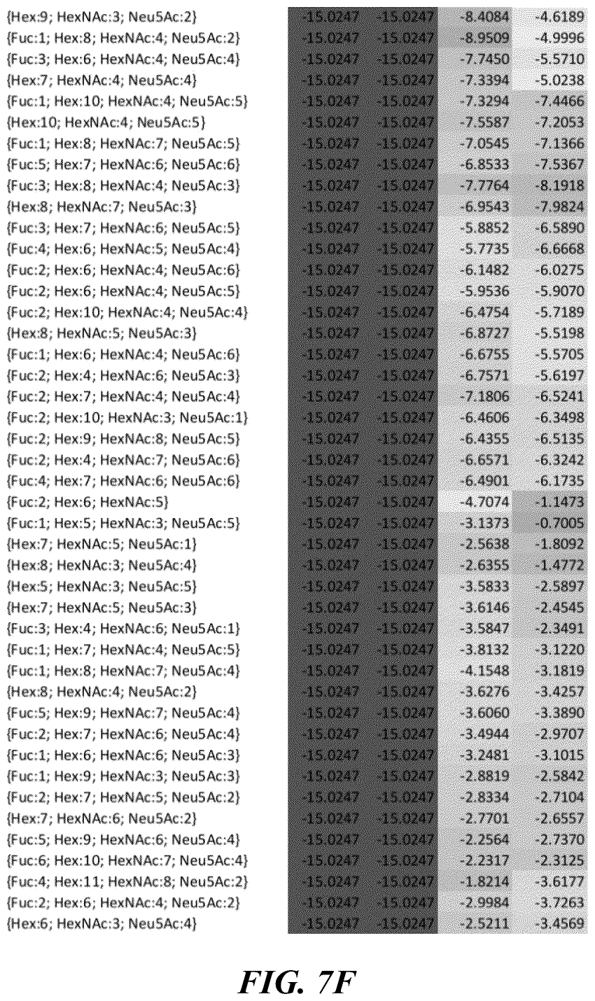

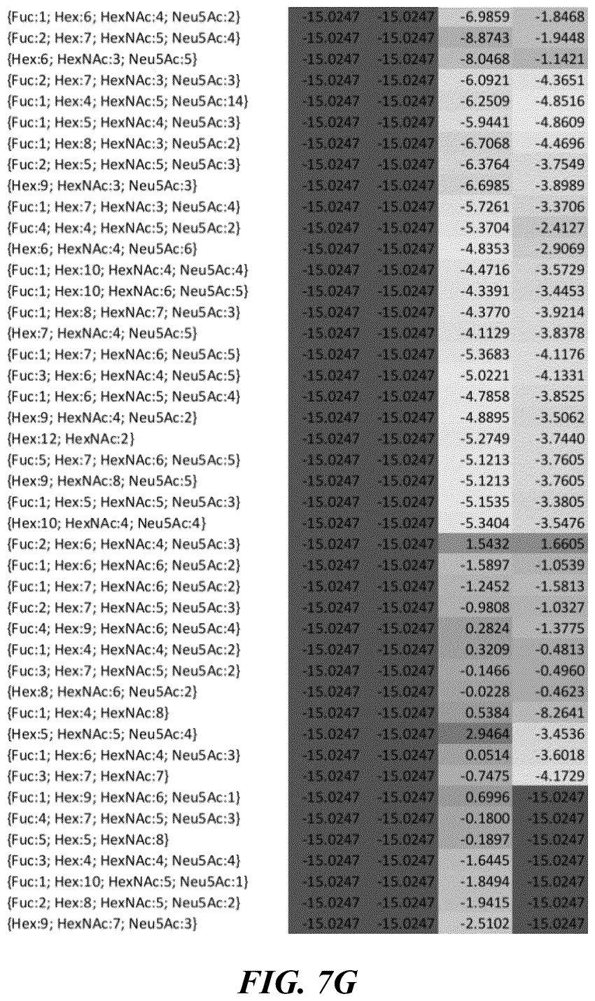

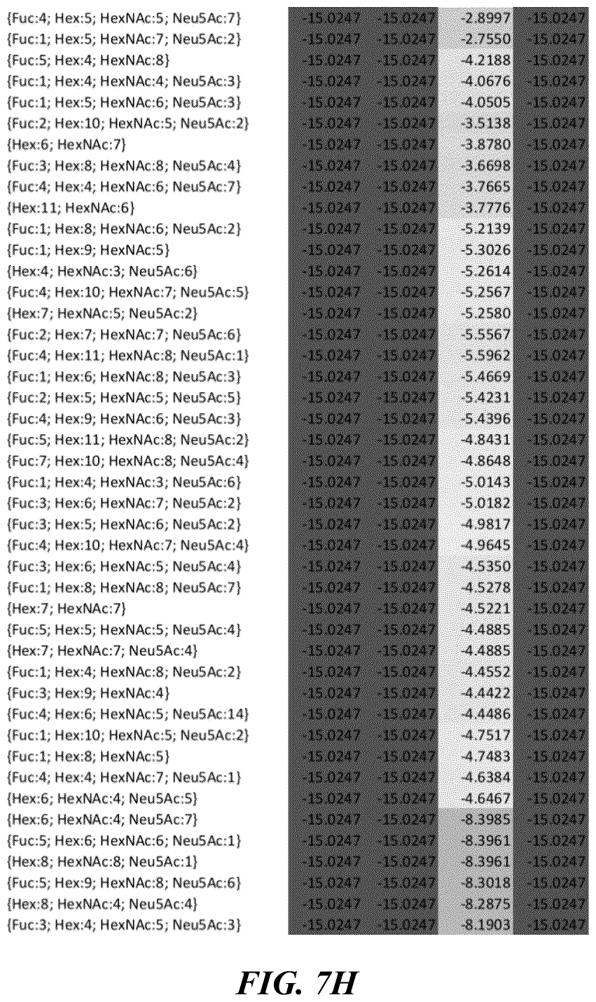

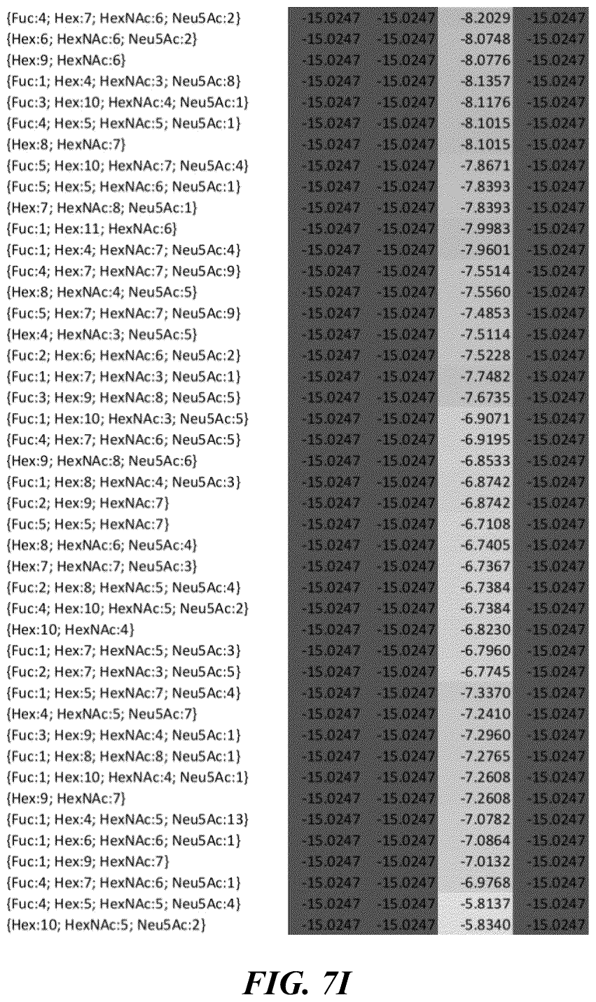

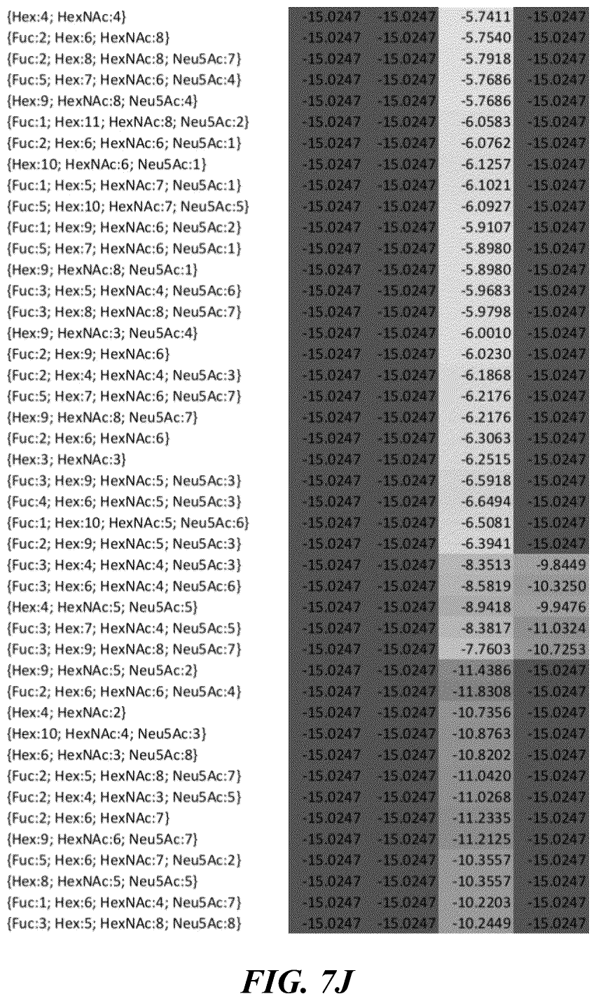

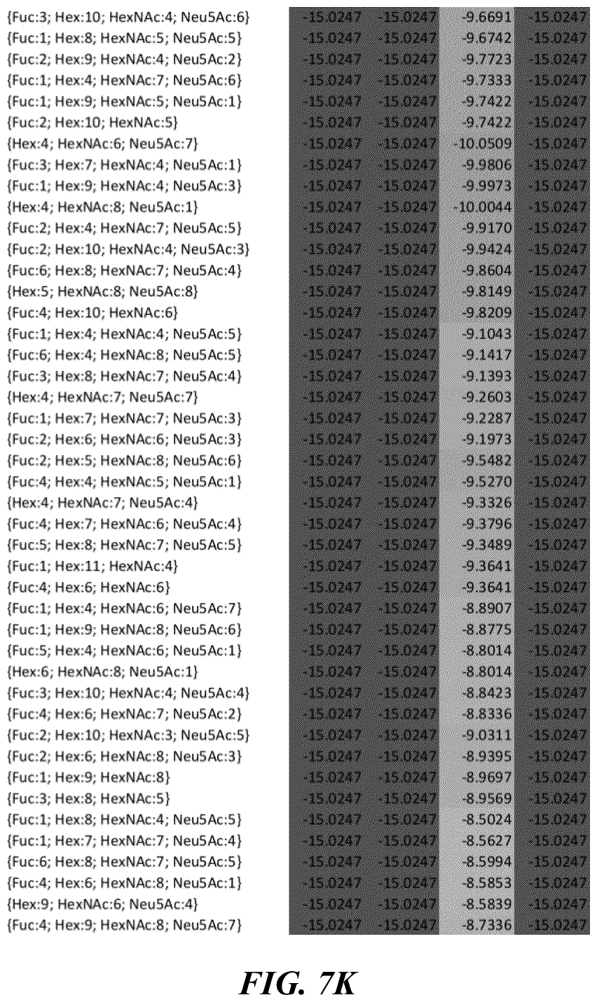

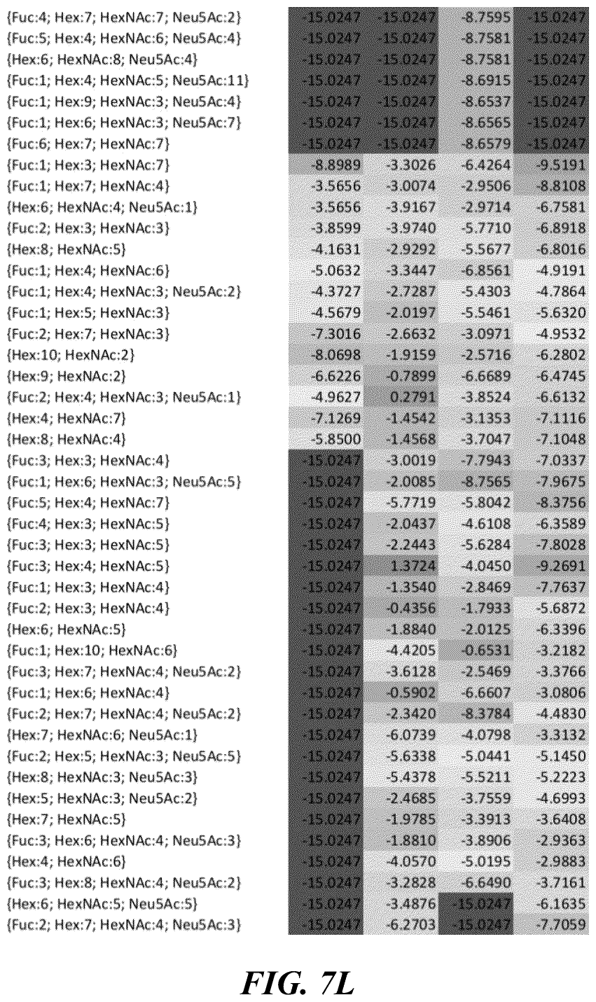

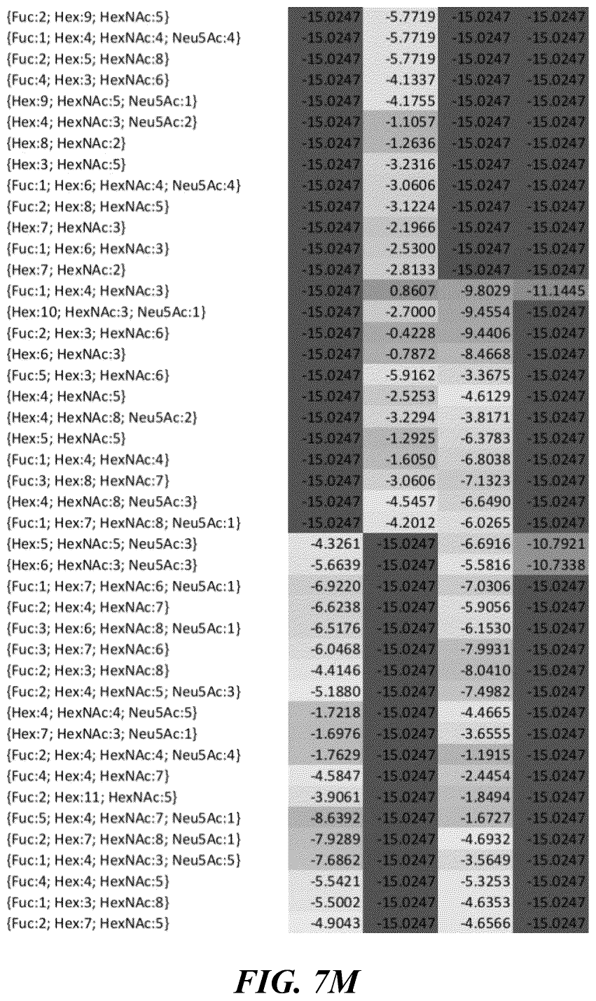

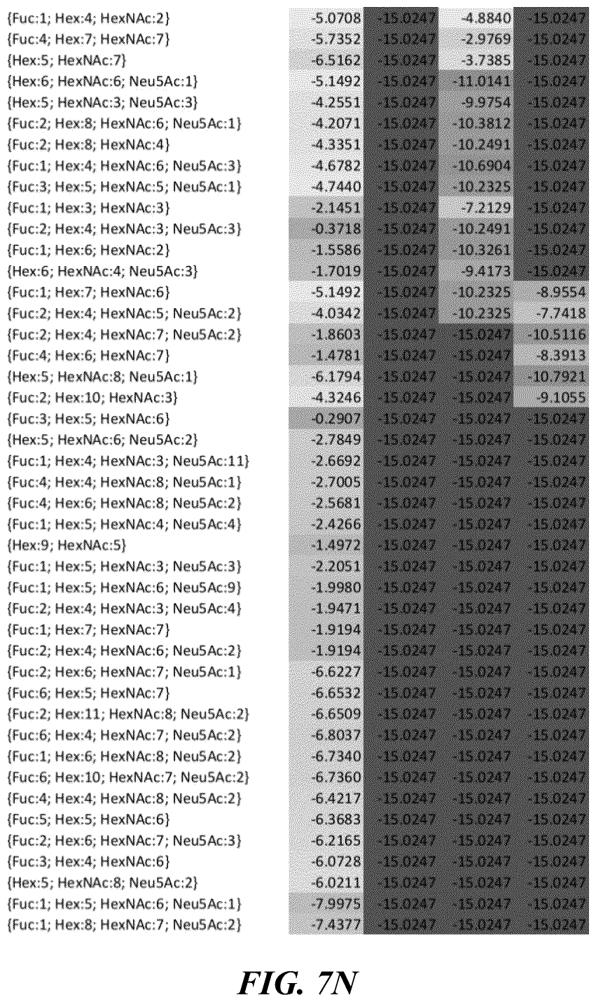

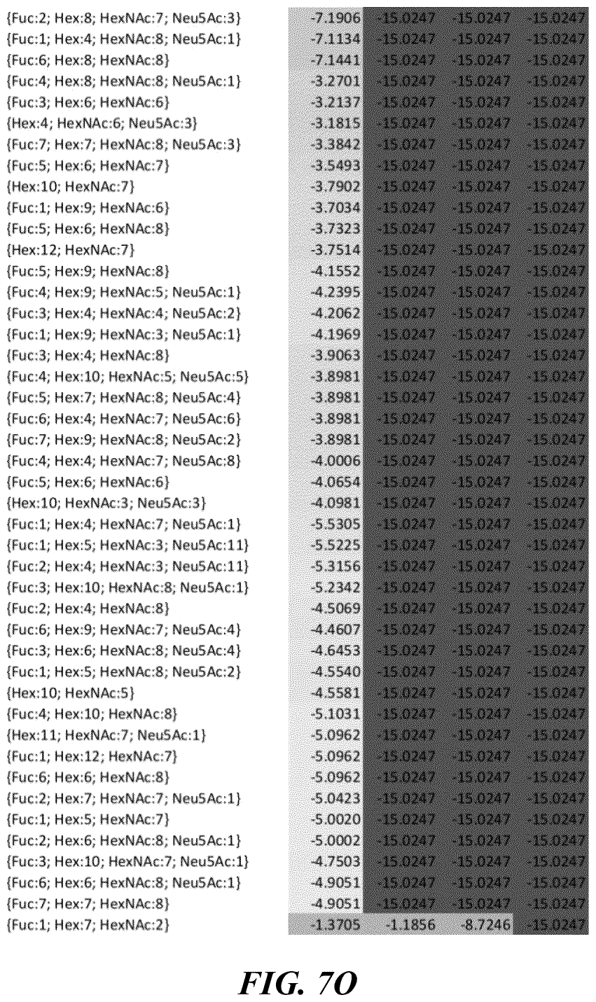

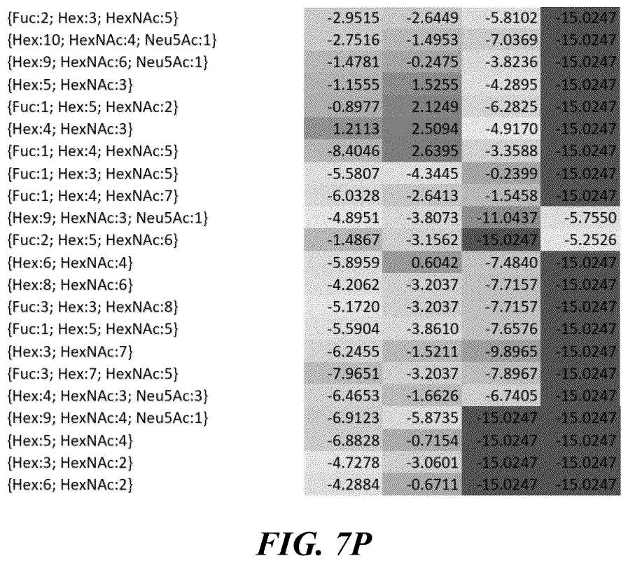

Differential N-glycan profiling of IgM, IgG, whole plasma, and EV isolates from blood. A qualitative and quantitative comparative analysis of N-glycans detected in the four types of analyzed blood-derived isolates (IgM, IgG, total plasma, and EVs) was conducted with an exhaustive list of 679 glycans, encompassing all the non-redundant N-glycan compositions identified in the four blood isolates. This differential analysis further demonstrated the uniqueness and high complexity of the four examined N-glycomes (FIG. 2J and FIGS. 7A-7P). IgM and IgG immunoglobulins as well as total plasma and plasma-derived EVs were clustered in two distinct clades, based on their respective N-glycome profiles (FIG. 2J). 68 N-glycans were uniquely detected in human serum IgM (FIG. 2K), among which 25% were highly fucosylated (i.e., 5-7 fucose residues) and 10% were highly sialylated (i.e., 5-11 SiA residues) N-glycans. 185 N-glycans were uniquely detected in total human plasma (FIG. 2K), among which 12% were highly fucosylated (i.e., 5-7 fucose residues) and 31% were highly sialylated (i.e., 5-14 SiA residues) N-glycans. In contrast, the numbers of N-glycans uniquely detected in human serum IgG and total EV isolate were relatively low (13 and 20 N-glycans, respectively, FIG. 2K). Unique IgG N-glycans did not exhibit high degrees of fucosylation and sialylation since highly fucosylated and sialylated glycans were not detected in IgG. A few N-glycans unique to EVs were highly sialylated (i.e., 5-14 SiA residues) N-glycans. Similarities were observed in the relative abundance levels of the N-glycans detected in the four analyzed blood-derived samples (FIGS. 7A-7P). Several glycans, including FA2G2S2, FA2BG2S2, A2G2S2, and A3G3S3, were detected at high abundance, and other glycans, including A4G4S4, FA4G4S4, A3G3S2, and FA3G3S2, were detected at medium abundance in each analyzed isolate sample. Other glycans, e.g., the set of fucosylated analogs of A3G3S3, exhibited different relative abundances in the four examined blood isolate types. As an illustration, FA3G3S3 was detected at lower abundance in the IgM and IgG isolates, compared to plasma and EVs. F2A3G3S3 was not detected in the IgG isolate and detected in relatively low abundance in the other three blood isolates. F3A3G3S3 was detected only in plasma and EV isolates at relatively low abundance, and F4A3G3S3 was detected only in total plasma. Several neutral glycans also exhibited significantly different abundance levels in the four types of the blood-derived isolates. For instance, Man10 was detected at much lower abundance in the IgM and EV isolates, compared to the IgG isolate and total plasma. Man9 was detected at higher abundance in the IgG isolate, compared to the other three blood isolates, while Man8 and Man7 were detected only in the IgG isolate (FIGS. 7A-7P). Overall, the above-described results demonstrate that the newly developed workflow is a powerful, straightforward, flexible, and highly sensitive approach to decipher the N-glycomes of complex biological samples. It allowed us to further expand the glycan coverage of the four analyzed blood-derived isolates, including human plasma-derived EVs, using minute amounts of samples, and identify glycan species unique to a specific type of a blood isolate.

### Single Mammalian Cell N-Glycome Profiling

Single-cell loading and in-capillary N-glycan release. Individual mammalian cells were introduced into the CE capillary in a controlled manner using a hydrodynamic injection mode (FIG. 1A). To visually confirm the cell loading process, the cells were treated with a plasma membrane-binding dye and subsequently imaged using bright-field and fluorescence microscopy techniques (FIGS. 1C-1D). Upon introduction into the capillary, the evaluated cells exhibited a tendency to weakly and transiently adhere to the capillary surface if the hydrodynamic flow was either halted or maintained at an excessively low rate. This phenomenon of cell immobilization is likely attributable to the formation of hydrogen bonds and van der Waals interactions between the silanol groups of the bare fused silica capillary surface and numerous chemical groups present on the cell surface. When the diameter of the injected cells was close to the internal diameter of the capillary, cells got slightly squeezed upon entering the capillary, and their size could further impede their mobility. This tendency was strategically exploited to facilitate cell stacking for the effective injection of several cells, as well as to better control the distance between the injected individual cells and the capillary inlet, thus making each injection more reproducible.

The cells loaded into the CE capillary were sandwiched between two plugs of a PNGase F digestion solution, and two short CE voltage pulses (30 s each) were applied in normal and reverse polarity to effectively mix the cells with the glycosidase. No lysis buffer was employed and/or injected to preserve the cell integrity and release only the N-glycans from the cell surface. Ideally, to preserve cellular integrity, the cells should be maintained in a buffer solution that closely mimics physiological pH and osmolarity. However, such buffers are typically incompatible with MS or CE analysis and may result in ionization suppression, adduct formation, and decreased separation performance phenomena. In this study, a stacking strategy was used to enhance the peak shape and intensity and improve the separation of the detected glycans. To enable this strategy, the cells were resuspended in 1 mM ammonium acetate pH 6.7, immediately prior to their loading into the CE capillary, and the commercial PNGase F enzyme was diluted 7-fold in water to highly decrease the salt concentration (see Methods section). Given that the cells were exposed to a low osmolarity environment during the deglycosylation step for N-glycan release, an assessment of the post-incubation cell integrity was conducted through the offline incubation of single cells for 1 h, using the conditions employed in the in-capillary sample processing workflow (i.e., the cells were sandwiched between two PNGase F plugs). Fluorescence imaging of the single cells prior to and after offline incubation in a small piece of capillary did not reveal discernible alterations in the cell morphology, size, or membrane integrity (FIG. 1D). To further check the cell viability, morphology, and membrane integrity under the selected in-capillary sample processing conditions, a suspension of HeLa cells stained with a fixable dead cell dye (see Methods section) was incubated for 1 h with PNGase F in 1 mM ammonium acetate pH 6.7. Based on microscopic visualization, it was estimated that >40% of the HeLa cells were still alive after the deglycosylation step with PNGase F. It was also observed that the majority of the dead cells exhibited a morphology very similar to that of the live cells, suggesting that the cell integrity could be preserved under our experimental conditions for N-glycan release. Therefore, it was concluded that the majority of detected and identified glycans were removed from the cell surface.

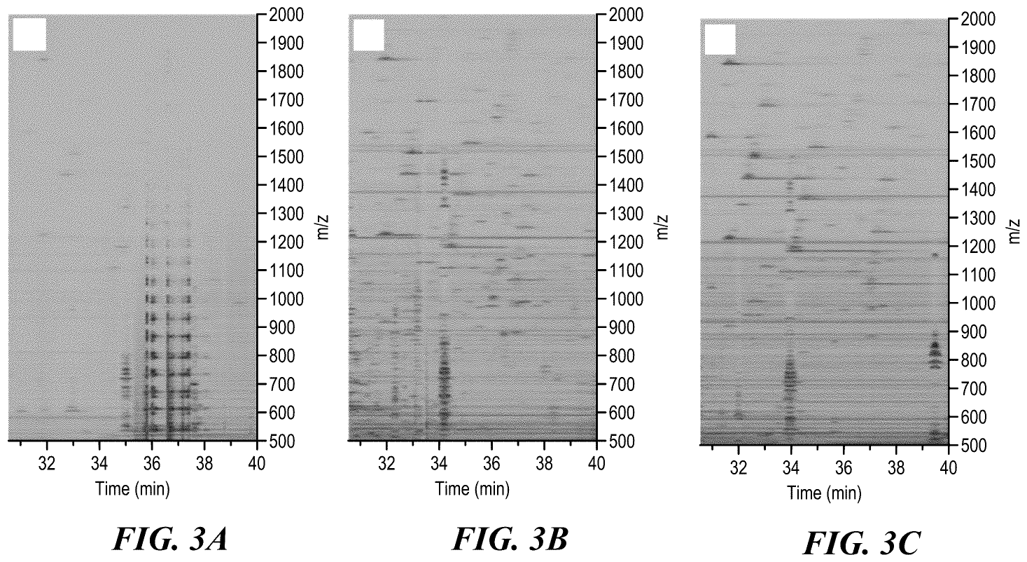

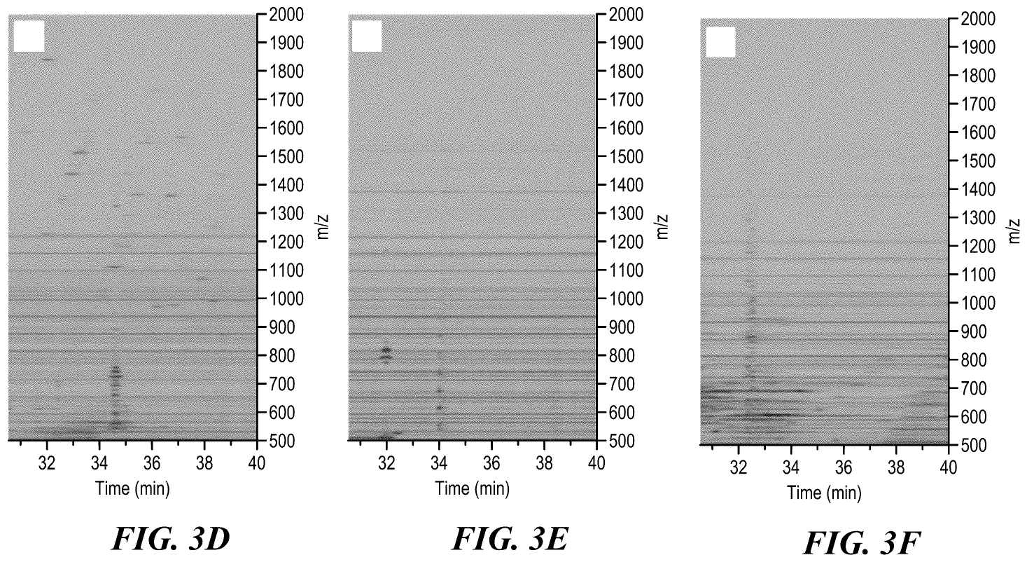

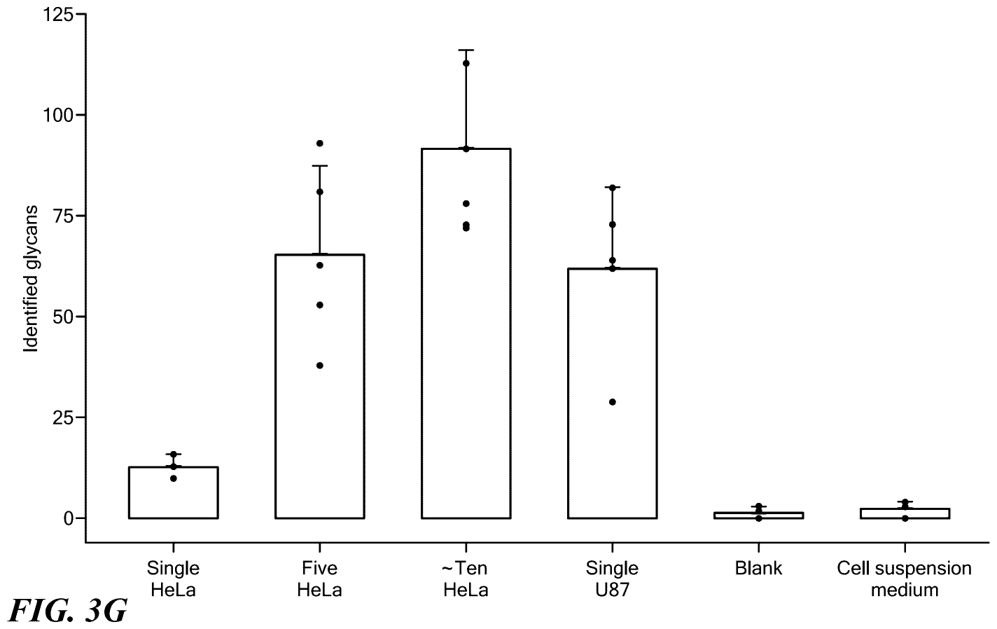

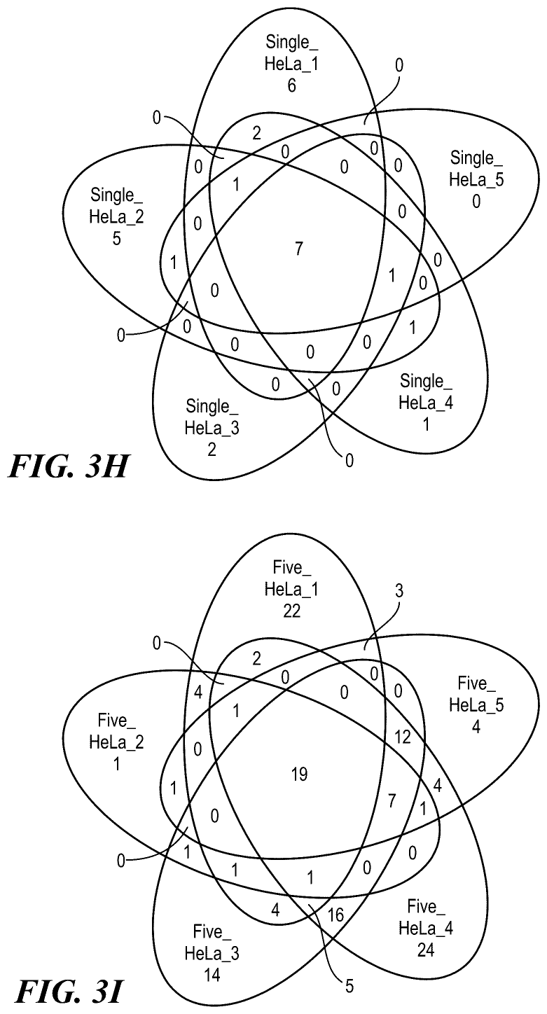

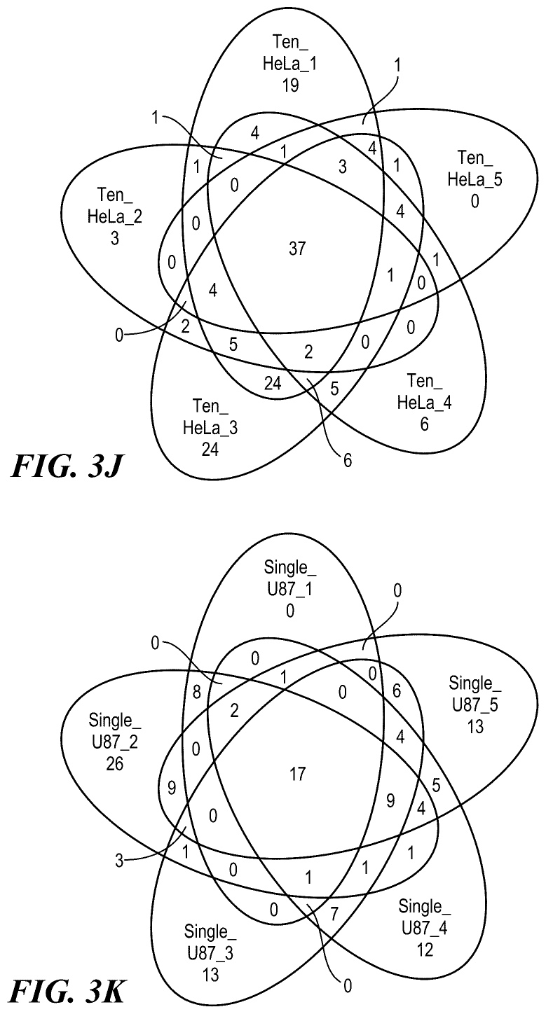

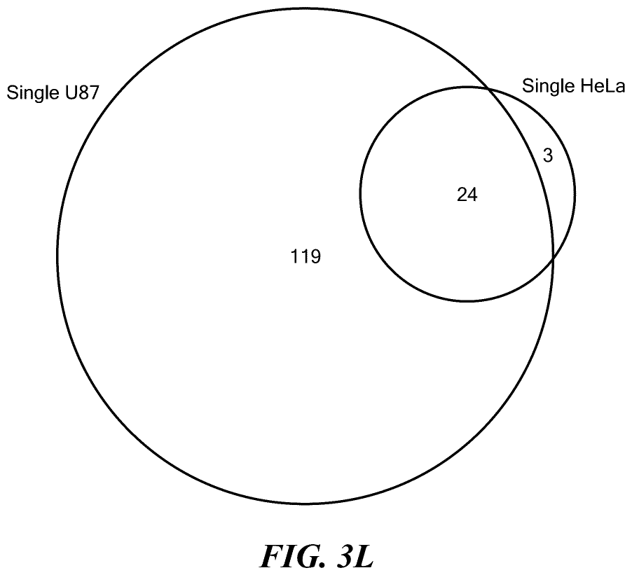

N-glycan profiling of single, five, and ˜ten HeLa cells. To assess the capability of the present method for direct and unbiased N-glycan profiling of mammalian cells, sets of five repetitive experiments were performed with the injection of one, five, and about ten (i.e., 10±3 cells, referred to as “bulk sample”) HeLa cells. Characteristic ion density maps acquired in CE-MS analysis of HeLa cell-derived N-glycans are presented in FIGS. 3A-3C. Processing of CE-MS¹ data resulted in the identification of 13±3 (one HeLa cell), 66±22 (five HeLa cells), and 92±24 (˜ten HeLa cells) N-glycans (n=5), respectively (FIG. 3G). Due to cell-to-cell heterogeneity (arising from, inter alia, molecular variability and cell-cycle position) and cell size variations (the surface areas of the injected HeLa cells being in the range 1,282-3,258 μm²), the number and type (i.e., monosaccharide composition) of identified N-glycans exhibited significant variations. FIGS. 3H-3J depict the overlap of the N-glycans detected in the five repetitive analyses acquired for each HeLa cell loading. In total, 27, 148, and 160 non-redundant N-glycan compositions were identified in one, five, and ˜ten HeLa cells, respectively. As shown in FIGS. 3E-3G, the levels of carryover derived from the analysis of preceding HeLa cell samples were insignificant while using the described capillary rinse cycles, based on control analyses performed with a water blank sample. CE-MS analyses of the cell suspension medium, collected from the same cell suspension used to inject individual cells, were also carried out. These experiments allowed confirmation that the detected glycans were not derived from extracellular proteins or contaminants present in the cell medium (FIGS. 3F-3G).

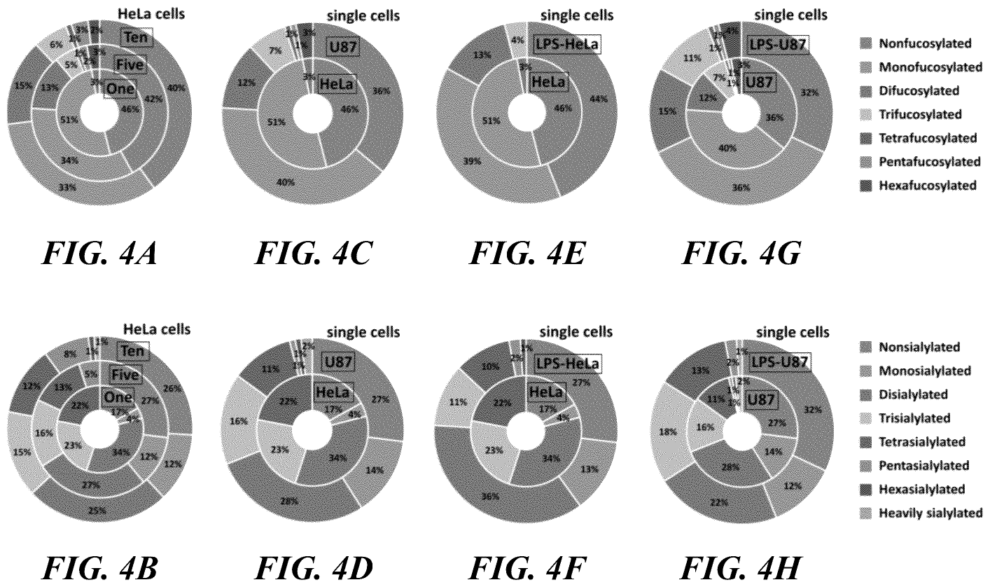

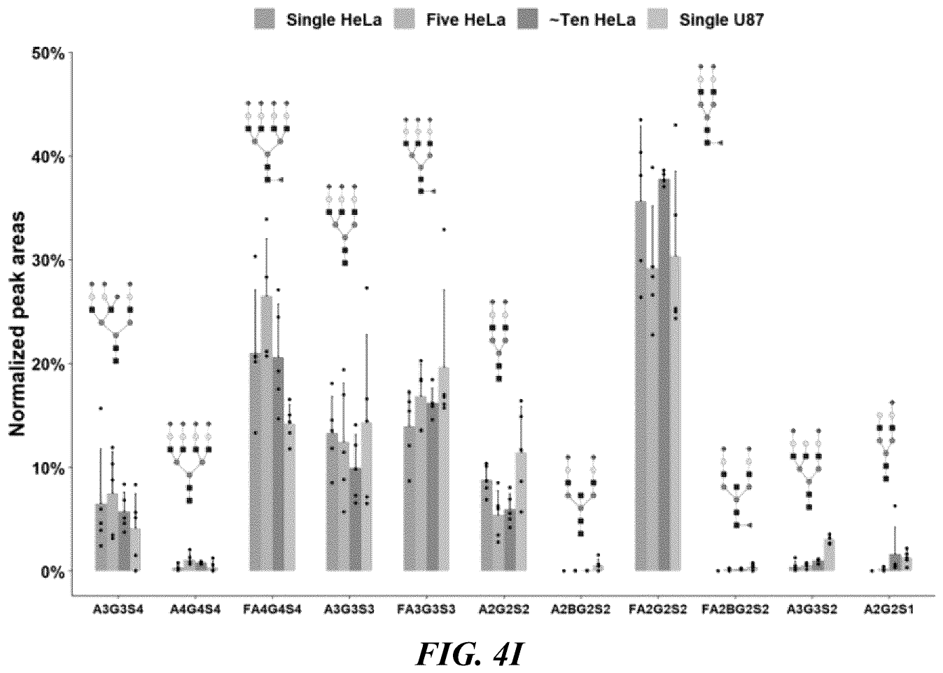

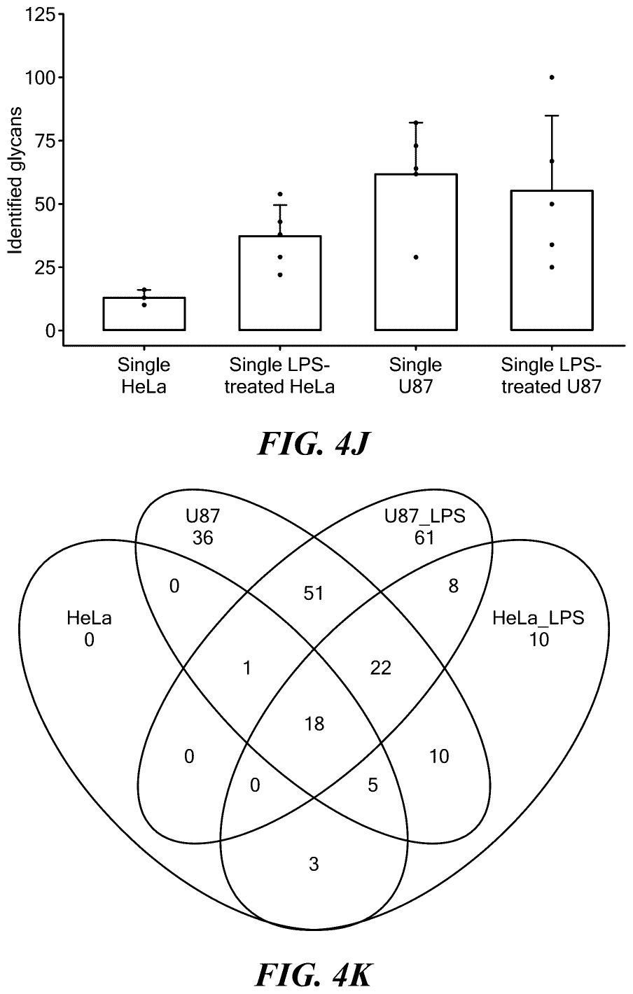

Higher levels of fucosylation (up to 6 fucose residues) and sialylation (5-12 SiA residues) were detected in 5-10 HeLa cells, compared to single HeLa cells, for which the degrees of fucosylation and sialylation of identified N-glycans did not exceed 2 and 4, respectively (FIGS. 4A-4B). These results indicated the extremely low abundance levels of highly fucosylated and highly sialylated N-glycans in HeLa cells since they could not be detected at the single HeLa cell level using the workflow described herein. Mono- and difucosylated N-glycans accounted for 51% and 3% of the total glycans detected in single HeLa cells, respectively (46% of glycans were nonfucosylated). For 5 and 10 HeLa cells, the fractional distributions of fucosylated N-glycans were 34%, 13%, 5%, 1%, 2%, and 3% (five HeLa), and 33%, 15%, 6%, 1%, 3%, and 2% (˜ten HeLa) for mono-, di-, tri-, tetra-, penta-, and hexafucosylated N-glycans, respectively (FIG. 4A). Mono-, di-, tri-, and tetrasialylated N-glycans accounted for 4%, 34%, 23%, and 22% of the total glycans detected in single HeLa cells, respectively (17% of glycans were nonsialylated). For 5 and 10 HeLa cells, the fractional distributions of sialylated N-glycans were 12%, 27%, 16%, 13%, and 5% (five HeLa), and 12%, 25%, 15%, 12%, 8%, 1%, and 1% (˜ten HeLa) for mono-, di-, tri-, tetra-, penta-, hexasialylated, and heavily sialylated (≥7 SiA residues) N-glycans, respectively (FIG. 4B).

Due to cell-to-cell heterogeneity, high variations in the absolute glycan abundances measured in the five repetitive analyses were observed for one, five, and ˜ten injected HeLa cells (e.g., for single HeLa cell measurements, the relative standard deviations (RSD) of peak areas could be as high as 65%). It was hypothesized that such significant variation might be mostly attributed to the cell size, surface area, and cell cycle state. Nevertheless, a substantial increase in the glycan abundance levels was demonstrated with increased loaded cell numbers. A linear relationship was demonstrated between the injected cell number and the total cellular glycan amount for the eight selected glycans, based on peak area measurements. FIG. 4E displays the normalized abundances of eleven representative N-glycans detected in one, five, and ˜ten HeLa cells (i.e., each glycan abundance was normalized with respect to the summed abundances of the eleven selected glycans). FA2G2S2, A3G3S3, FA3G3S3, and FA4G4S4 were detected at high abundance levels in the analyzed HeLa cells. To the contrary, A2G2S1, A2BG2S2, FA2BG2S2, A3G3S2, and A4G4S4 were measured at low abundances. Overall, the relative abundances of the eleven selected HeLa cell-derived N-glycans were consistent across different cell loading levels, based on peak area measurements. For instance, the relative abundance of FA4G4S4 was approximately twice lower than that of FA2G2S2 but about three times higher than that of A3G3S4 in one, five, and ˜ten HeLa cells.

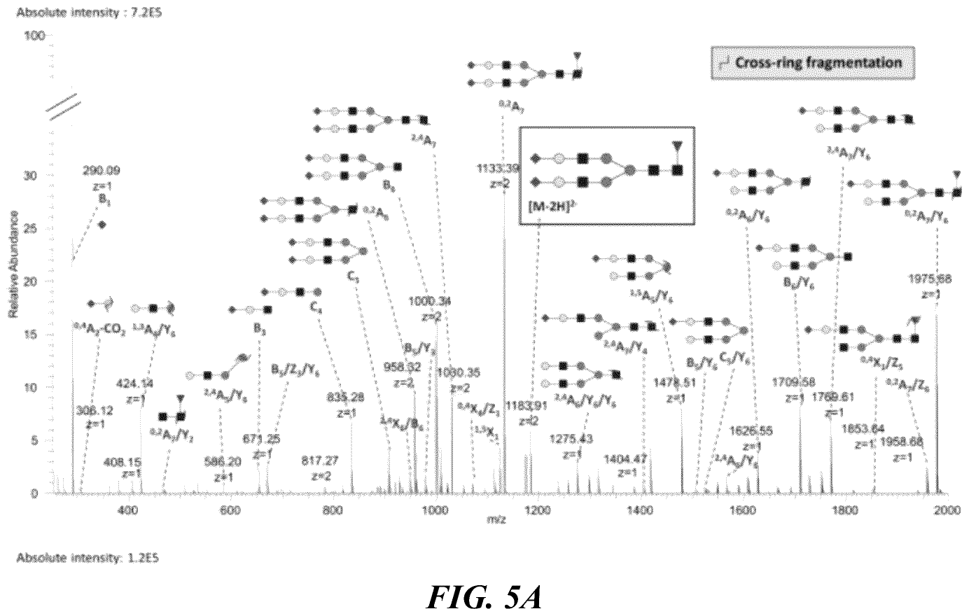

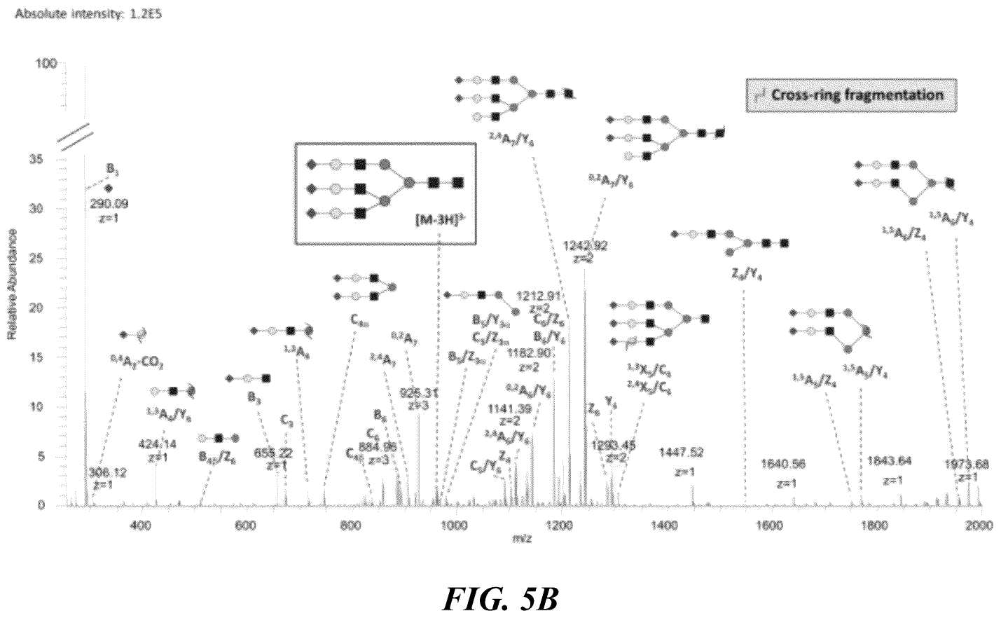

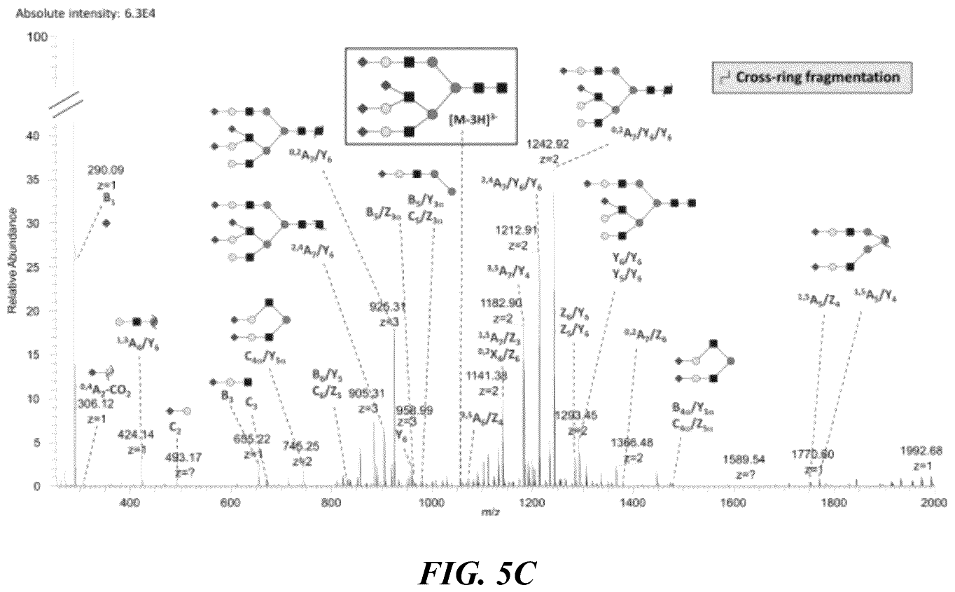

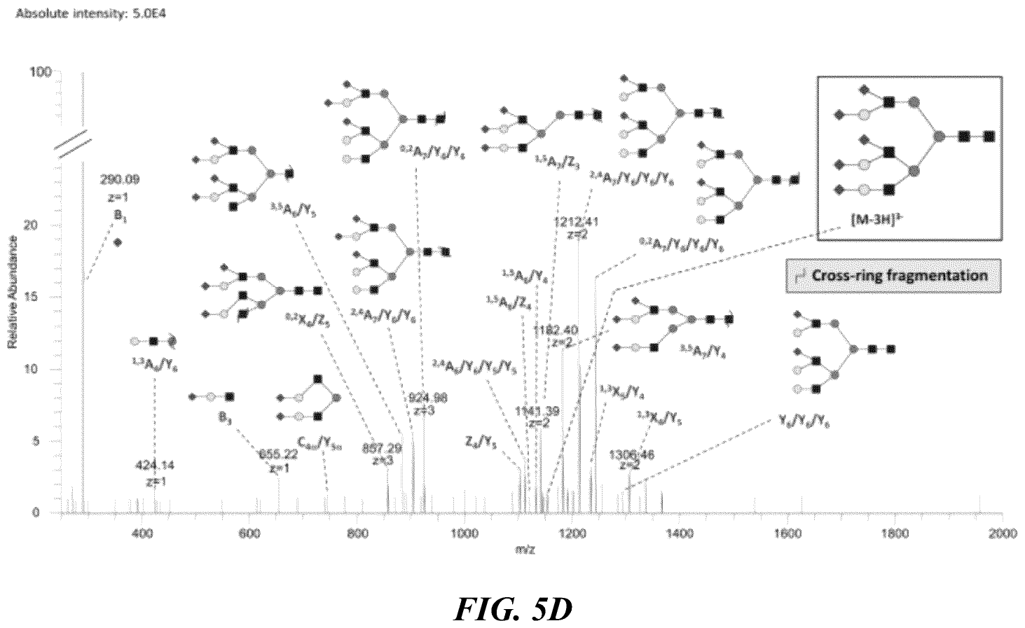

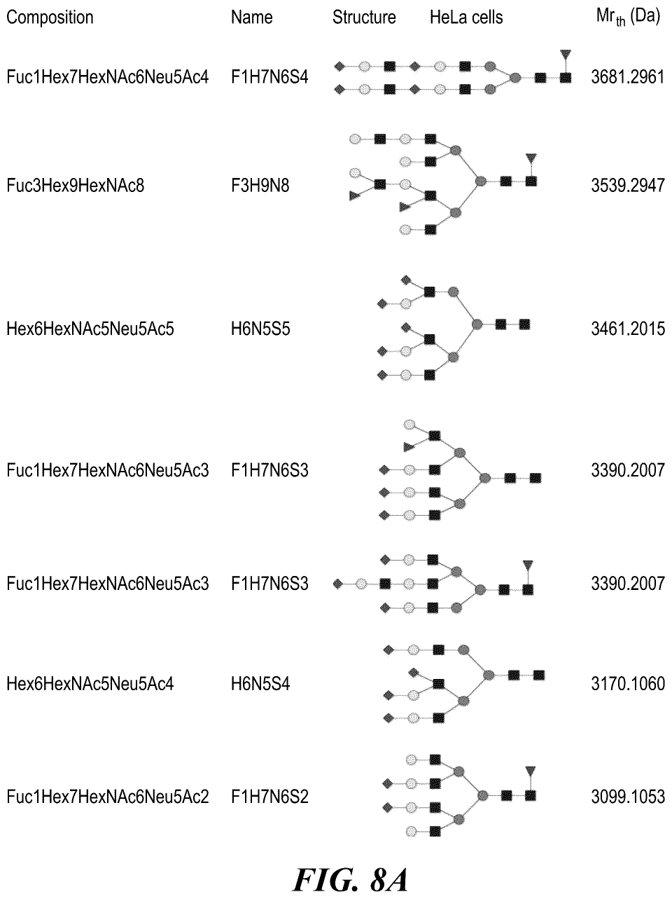

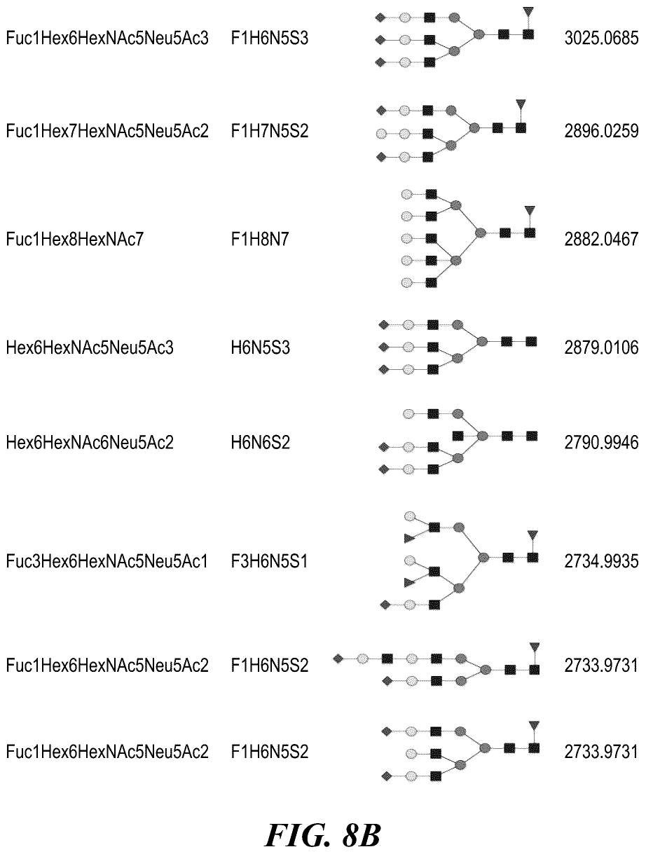

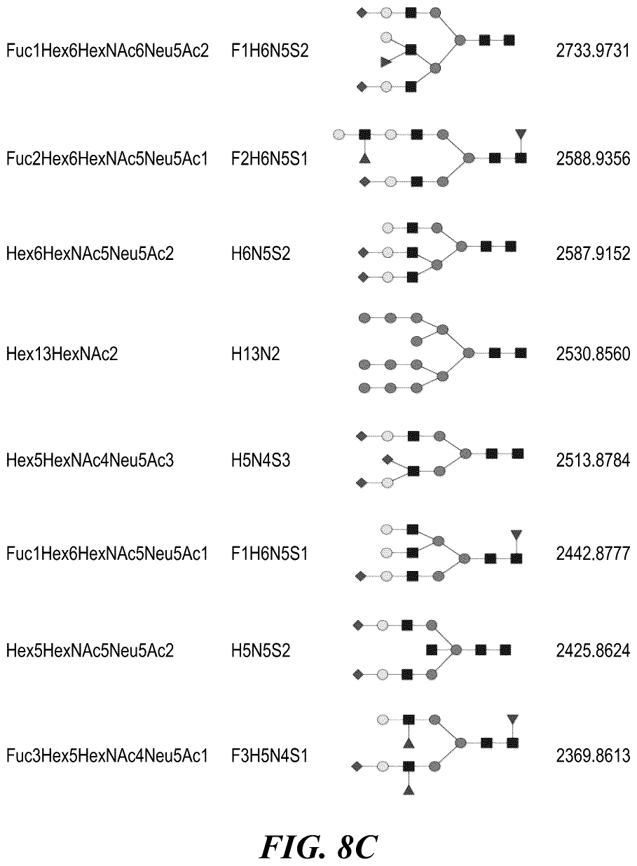

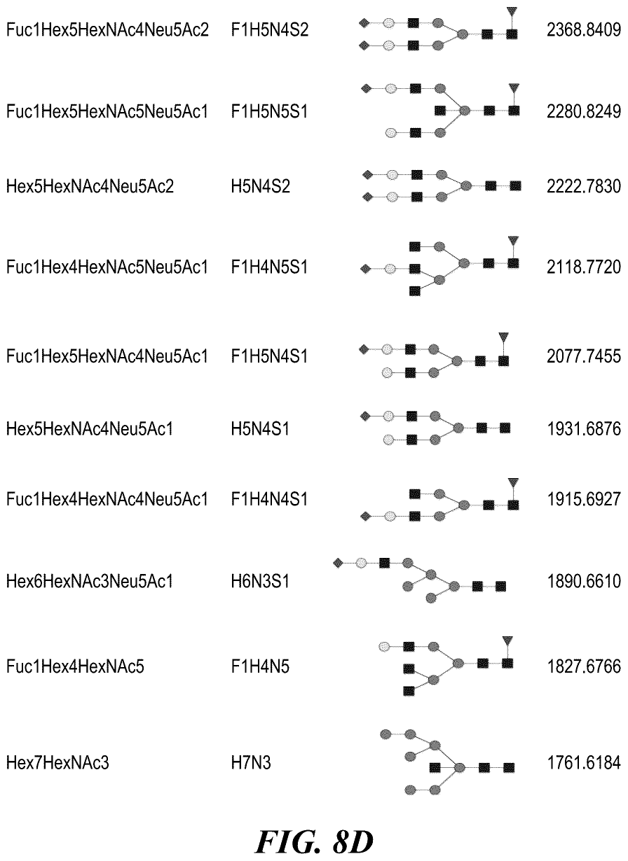

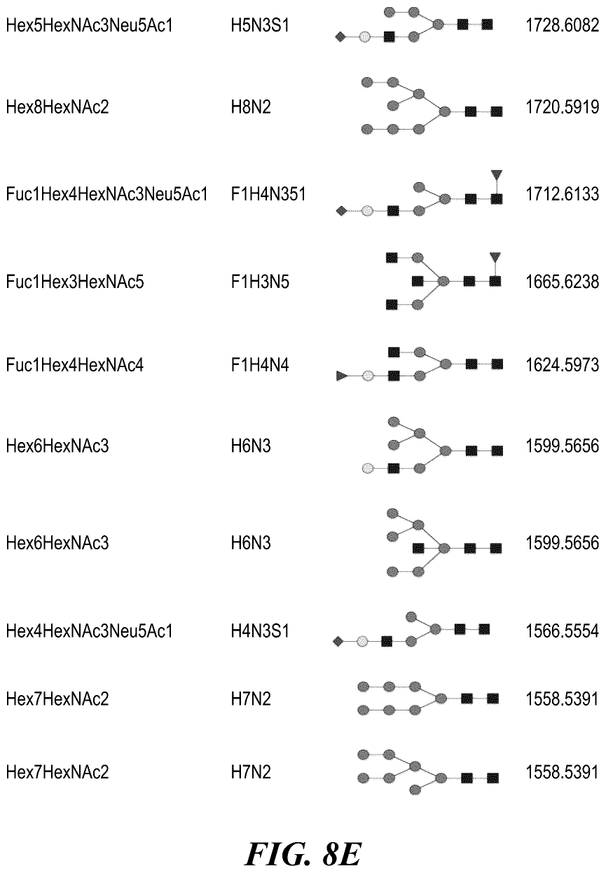

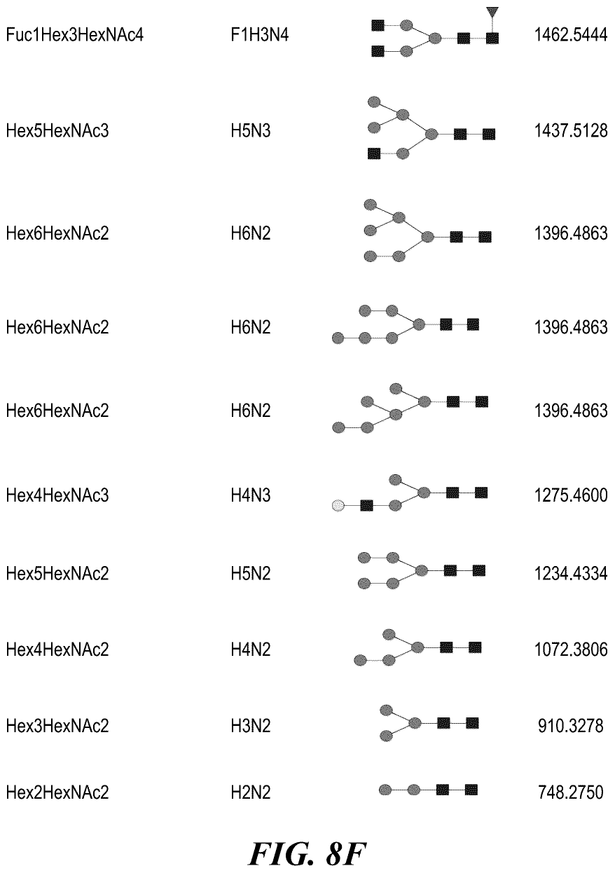

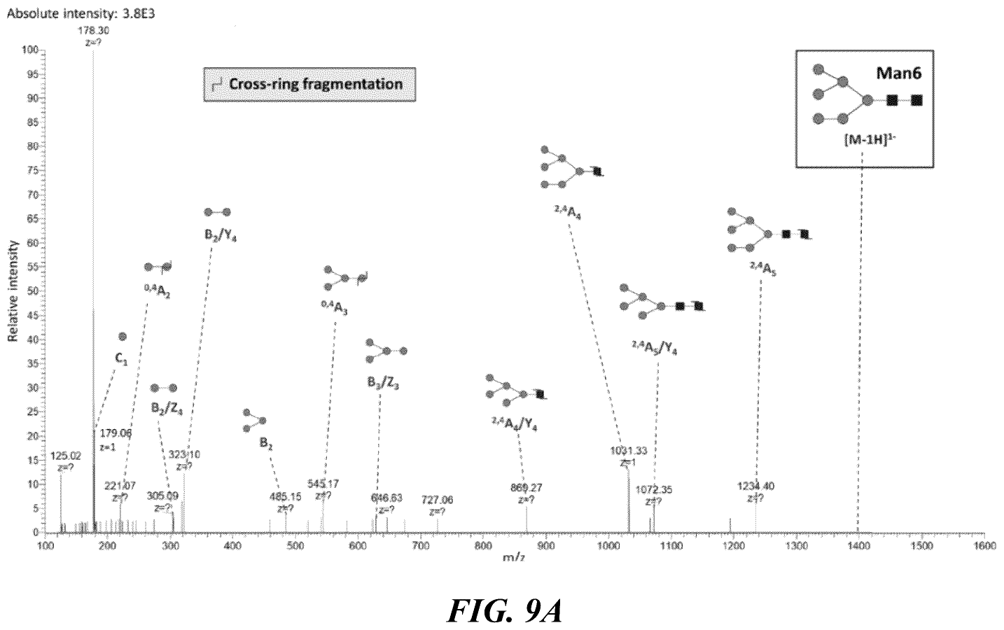

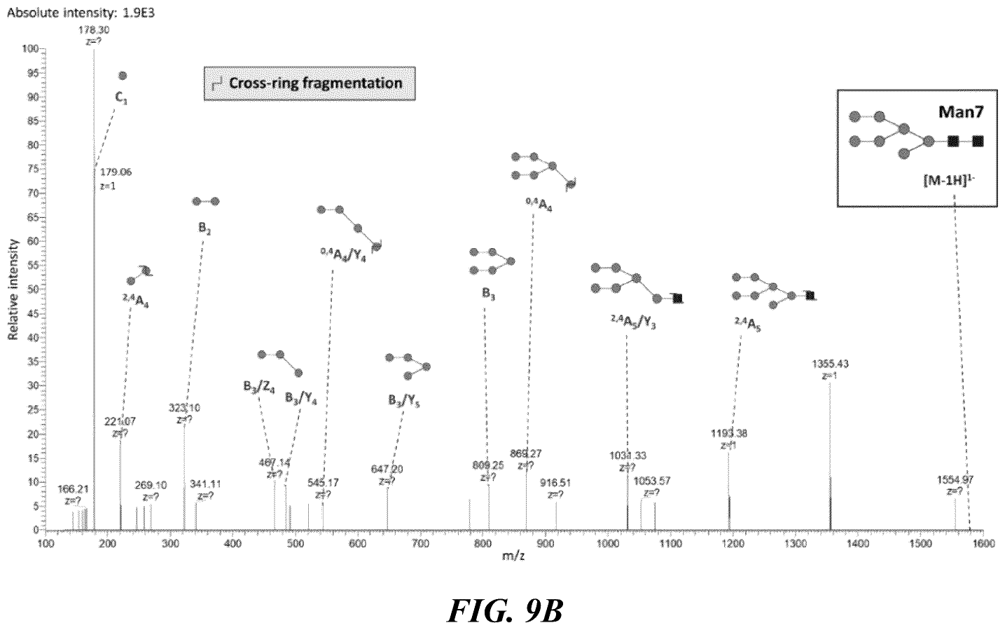

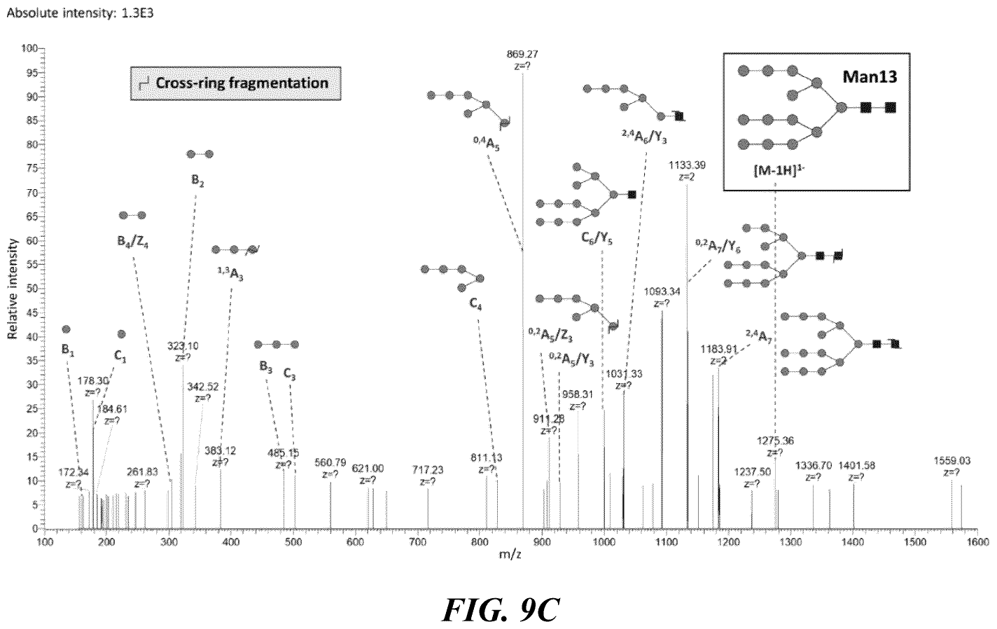

CE-MS² analyses of HeLa cell-derived N-glycans were performed to confirm the N-glycan composition identification results and provide information on structural features of the detected glycans (e.g., antenna-branching, fucose position, and SiA linkage). CE-MS² experiments performed with ˜ten HeLa cells resulted in accurate and unambiguous structural characterization of 53 N-glycans in HeLa cells, among which acidic (i.e., sialylated) and neutral glycans (FIGS. 8A-8F). As expected, neutral glycans (mobilized under the applied electric field through ion-dipole interactions with acetate anions present in the BGE⁴⁰) migrated later than sialylated glycans. FIGS. 5A-5D show characteristic MS² spectra of three representative sialylated N-glycans detected in HeLa cells. The molecular ions at m/z 1,183.42, 958.66, and 1,055.69 were selected as precursor ions for the MS²-based structural characterization of FA2G2S2 (Mrₜh 2,368.84 Da, FIG. 5A), A3G3S3 (Mrₜₕ 2,879.01 Da, FIG. 5B), and A3G3S4 (Mrₜₕ 3,170.11 Da, FIG. 5C), respectively. The MS² spectra of these sialylated glycans all exhibited a predominant fragment ion B₁¹⁻ at m/z 290.09, corresponding to the loss of one SiA residue at the termini of the antennae, and the diagnostic ion ^{0,4}A₂-CO₂¹⁻ at m/z 306.12, revealing the presence of α-2,6 5-N-acetylneuraminic acid (Neu5Ac) linkages 54. In the mass spectra of the fucosylated FA2G2S2 glycan, the characteristic mass difference of 206.08 Da between ^{2,4}A₇²⁻ (m/z 1,029.85) and ^{0,2}A₇²⁻ (m/z 1,132.89), and ^{2,4}A₇/Y₆¹⁻ (m/z 1,769.61) and ^{0,2}A₇/Y₆¹⁻ (m/z 1,975.68) ion pairs located the fucose on the chitobiose core^{40,55}. For the tri-antennary A3G3S3 glycan, the diagnostic ions B₅/Z_{3α}¹⁻ (m/z 961.31), B₅/Y_{3α}¹⁻ (m/z 979.32), and C_{4α}²⁻ (m/z 745.25) allowed us to assign a branched 3-linked antenna^{40,55}. Similarly, the fragment ions B₅/Z_{3α}¹⁻ (m/z 961.31), B₅/Y_{3α}¹⁻ (m/z 979.32), and C_{4α}/Y_{5α}²⁻ (m/z 745.25) were diagnostic of a branched 3-linked antenna for the tri-antennary A3G3S4 glycan. The pentasialylated glycan A3G3S5, a complex glycan that was rarely structurally characterized by tandem MS in the past⁴⁰, was also detected in HeLa cells (see below and FIG. 5D for the MS² fragmentation pattern of this glycan). CE-MS² data also allowed structural characterization of neutral N-glycans in HeLa cells (FIGS. 8A-8F), including high mannose-type N-glycans in HeLa cells, from Man2 to Man13, with or without a bisecting GlcNAc residue (FIGS. 8A-8F). FIGS. 9A-9C depict the fragmentation patterns of Man6, Man7, and Man13. For these glycans, characteristic fragment ions (e.g., at m/z 179.06, 323.10, 485.15, 869.27, and 1,031.33) were supportive of a branched oligomannosyl structure 56.

Differential N-glycome analysis of single HeLa and U87 cells. Next, the in-capillary workflow was applied to the CE-MS analysis of single U87 cells to assess, as a proof-of-concept, whether qualitative and/or quantitative differences in cell surface N-glycomes of different cell types could be detected at the single-cell level. In comparison to single HeLa cells, a significantly higher number (˜5-fold) of N-glycans were detected and identified in single U87 cells. Five repetitive experiments carried out with single U87 cells resulted in the detection of 62±20 N-glycans per single cell (FIGS. 3D-3G). In total, 143 non-redundant N-glycan compositions were identified in the analyzed single U87 cells (FIG. 3K). The examination of HeLa and U87 cell-containing droplets under a bright-field microscope showed that the two cell lines exhibited similar morphological characteristics in suspension but distinct size distributions. The average diameters and surface areas of representative HeLa and U87 single cells were determined to be 21.8±4.9 μm and 1,563±763 μm² (HeLa), and 26.1±6.6 μm and 2,282±1,266 μm² (U87), respectively. The higher number of N-glycans detected in single U87 cells could, therefore, be attributed in part to the larger size of this cell type (˜2-fold higher surface area), compared to HeLa cells. Yet, the non-linear relationship between the number of detected glycans and the size of the analyzed mammalian cells indicated that, as expected, other factors, e.g., unique molecular features and their abundances specific to HeLa and U87 cells, contributed to the significantly different numbers of N-glycans detected at the surface of HeLa and U87 single cells. Besides, the intrinsic cell morphology and structural characteristics of each cell type might also create locus-dependent steric hindrances at the surface of the single cells, resulting in differential accessibility of PNGase F to these specific cell surface loci.

89% of the glycans identified in single HeLa cells were also detected in single U87 cells (FIG. 3L). These commonly detected glycans accounted for 17% of the total number of N-glycans identified in single U87 cells, indicating that 83% of the glycans detected in U87 cells were specific to this cell line or could not be detected in HeLa cells due to their very low abundance or under-representation in single HeLa cells. In single U87 cells, higher levels of fucosylation (up to 6 fucose residues) and sialylation (up to 12 SiA residues) were detected, compared to single HeLa cells (FIGS. 4C-4D). Consequently, the unique glycans detected in single U87 cells encompassed tri-, tetra-, penta-, and hexafucosylated glycans, and sialylated glycans composed of 5-12 SiA residues, which were not detected in single HeLa cells. In single U87 cells, mono-, di-, tri-, tetra-, penta-, and hexafucosylated N-glycans accounted for 40%, 12%, 7%, 1%, 1%, and 3%, respectively (36% of glycans were nonfucosylated, FIG. 4C). The fractional distributions of sialylated N-glycans were as follows: 14% (mono-), 28% (di-), 16% (tri-), 11% (tetra-), 1% (penta-), 1% (hexa-), and 2% (heavily sialylated, i.e., ≥7 SiA residues), respectively (27% of glycans were nonsialylated) (FIG. 4D).

Noticeable differences in the abundances of the N-glycans detected in HeLa and U87 single cells were also observed, based on peak area measurements. As an illustration, FIG. 4I shows the relative abundances of eleven selected N-glycans commonly detected in HeLa and U87 single cells. Based on the statistical paired t-test, a couple of glycans were detected in significantly higher or noticeably higher abundance levels in U87 cells, e.g., FA2BG2S2, A3G3S2, and A2G2S1 glycans (p<0.05), and FA3G3S3 and A2BG2S2 (p=0.1). On the contrary, the tetrasialylated glycan FA4G4S4 exhibited a higher abundance in HeLa cells (p=0.06).

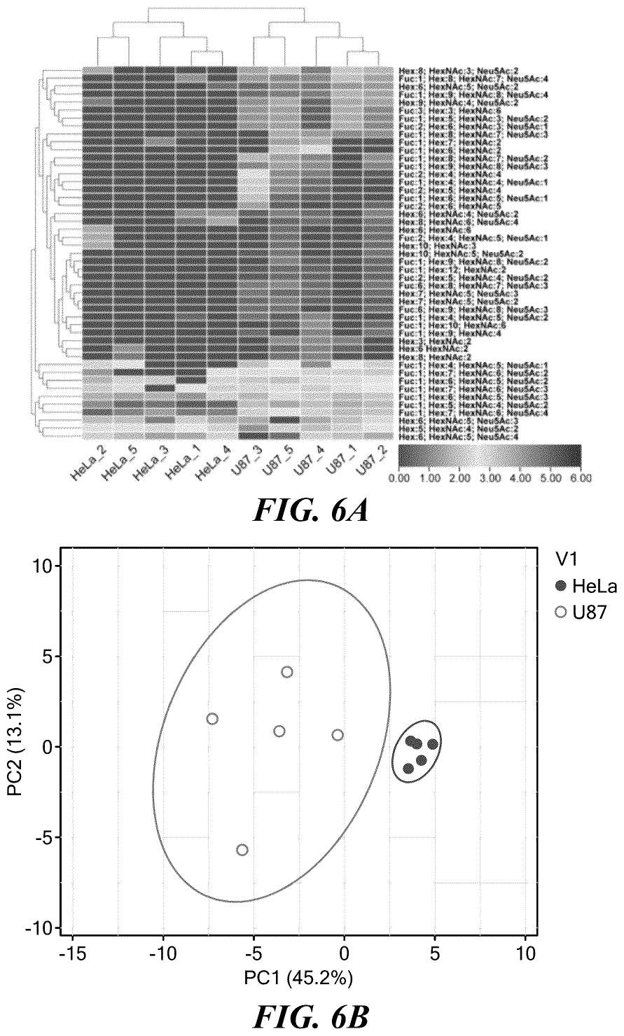

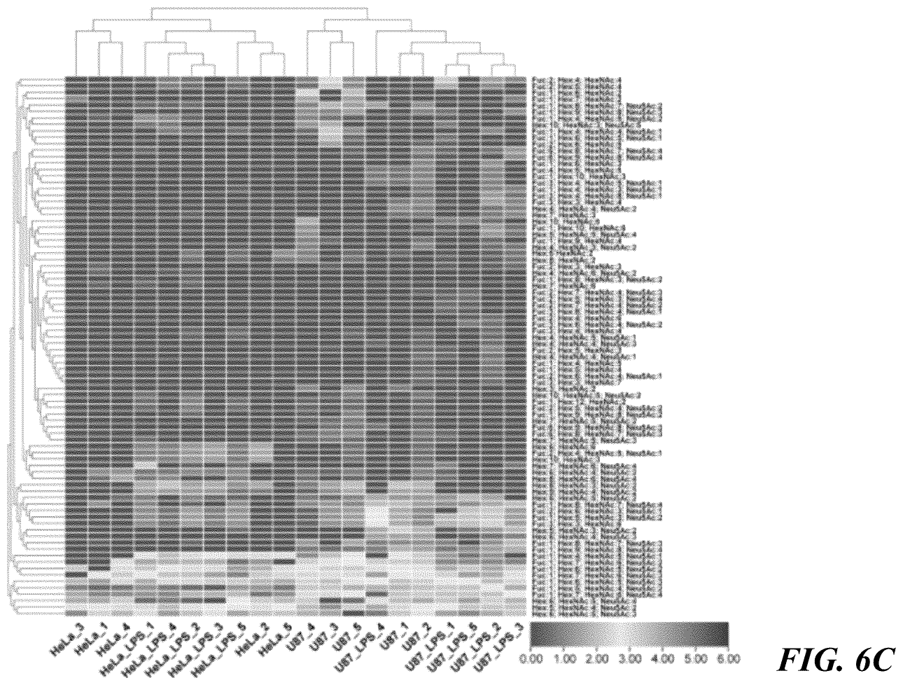

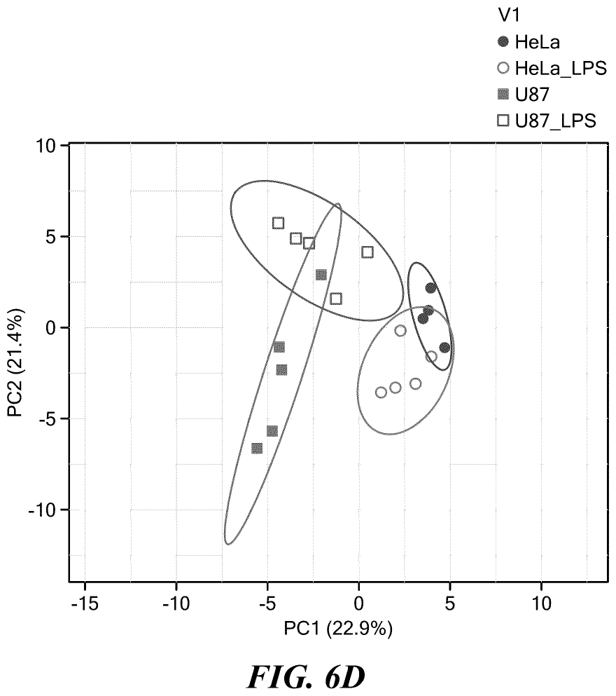

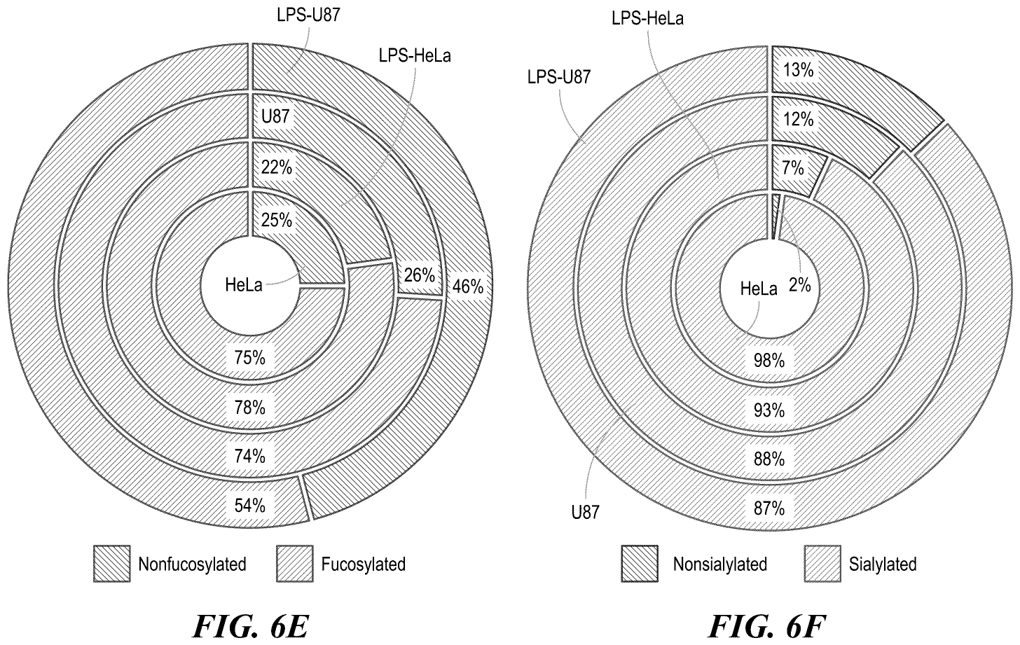

FIG. 6A depicts the results of non-supervised Euclidean distance-based hierarchical clustering of quantitative glycomic profiles of 47 representative N-glycans detected in HeLa and U87 single cells (i.e., glycans that were detected in at least three CE-MS analyses out of the ten total repetitive analyses). This clustering analysis yielded a reasonable differentiation of HeLa and U87 cell lines, according to their respective single-cell N-glycome profiles. As shown in FIG. 6A, HeLa and U87 single cells were clustered into two distinct clades, based on five repetitive analyses acquired for each single-cell type. Glycans like FA2G2S2, FA3G3S3, and FA4G4S4 were clustered into the same clade due to relatively high intensity levels in both HeLa and U87 cell types. Other clades reflected similar intensity levels of relatively medium abundance glycans, e.g., FA3G3S2 and FA4G4S3, in the two analyzed cell types. Interestingly, Man3 and the fucosylated analogs of Man6 and Man12 were only detected in single U87 cells and not in single HeLa cells, while Man6, Man8, and FMan7 were detected in both cell types at similar intensity levels but with a higher frequency in single U87 cells. The previously reported studies on glycomic analysis of U87 or other cells focused on specific proteins' glycosylation and its biological significance did not provide information on cell surface glycans' abundances⁵⁷⁻⁵⁹. In contrast, the results disclosed herein present an inventory of single HeLa and single U87 cell surface glycans with their respective glycan compositions and abundances, which allows us to highlight significant and potentially biologically-relevant glycosylation differences between the two cell lines. Principal component analysis (PCA) was also conducted to visualize the dominant trends and underlying specific patterns in the datasets generated by HeLa and U87 single-cell analyses (FIG. 6B). Selecting the 47 representative N-glycans detected in HeLa and U87 single cells above-described (FIG. 6A), two distinct clusters could be clearly differentiated with PCA, corresponding to HeLa and U87 cell lines, respectively (FIG. 6B). The PCA algorithm, therefore, allowed us to confirm the uniqueness and specificity of HeLa and U87 single-cell N-glycomes.

Finally, proof-of-concept CE-MS² experiments were performed with ˜ten U87 cells and resulted in accurate and unambiguous structural characterization of 29 N-glycans, including sialylated and neutral N-glycans. FIG. 5D shows a characteristic MS² spectrum of A3G3S5, detected at a relatively medium abundance in U87 cells. The MS²-based structural characterization of this pentasialylated glycan was based on the [M−3H]³⁻ precursor ion at m/z 1,152.73. For this glycan, the detection of the C_{4α}/Y_{5α}²⁻ fragment ion at m/z 745.25 and the absence of C_{4β}/Y_{5β}¹⁻ ion at m/z 835.28 allowed the assignment of a branched 3-linked antenna⁴⁰. The typical mass difference of 60.02 Da between the pair of ions ^{2,4}A₇/Y₆/Y₆³⁻ (m/z 904.97) and ^{0,2}A₇/Y₆/Y₆³⁻ (m/z 924.98), and ^{2,4}A₇/Y₆/Y₆/Y₆²⁻ (m/z 1,212.41) and ^{0,2}A₇/Y₆/Y₆/Y₆²⁻ (M/z 1,242.42) confirmed the absence of a core fucose 55. Interestingly, ^{0,4}A₂-CO₂¹⁻ diagnostic ion at m/z 306.12 was missing despite the relatively high intensity of B₁¹⁻ ion at m/z 290.09 (i.e., compared to the intensity level of B₁¹⁻ ion detected in A3G3S4 fragmentation spectrum (FIG. 5C), in which ^{0,4}A₂-CO₂¹⁻ ion could be detected). These results revealed the presence of α-2,3 SiA linkages in A3G3S5.

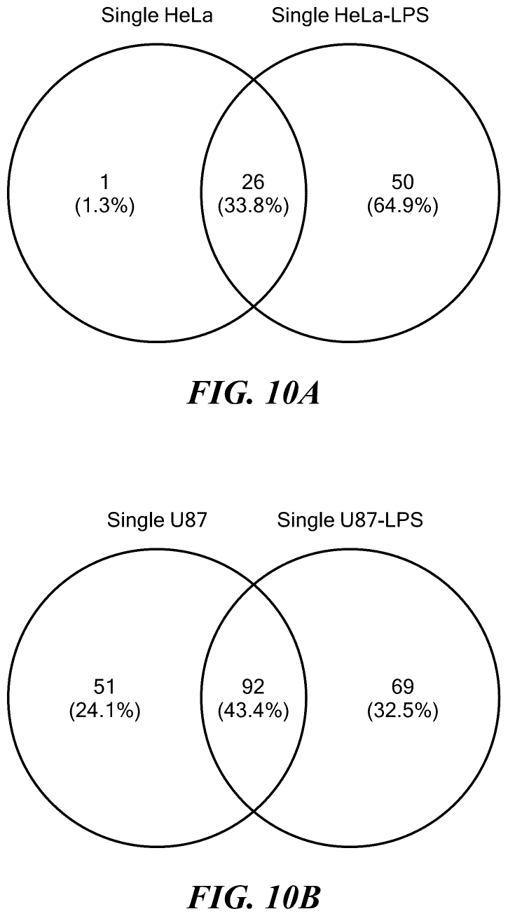

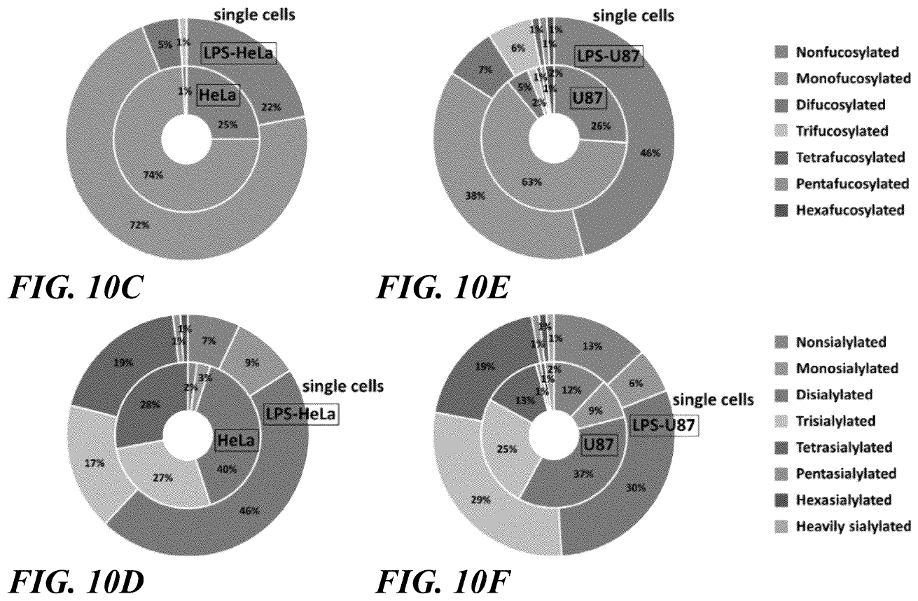

Single-cell N-glycome alterations induced by LPS stimulation. Previous studies reported that THP-1 mammalian cells treated with lipopolysaccharide (LPS) exhibited increased⁶⁰ or decreased⁶¹ levels of sialylation. Downregulation of glycan fucosylation was also reported for LPS-stimulated brain cells⁶². To test whether the developed CE-MS-based workflow could detect glycome alterations at the single-cell level, HeLa and U87 cells were stimulated with LPS. Interestingly, N-glycan profiling of single HeLa cells, after stimulation of HeLa cells with LPS, resulted in an ˜3-fold increased number of detected N-glycans, compared to the untreated HeLa cells (FIG. 4J). On average, 37±12 N-glycans per single HeLa cell (n=5) were identified after LPS treatment, and 76 non-redundant N-glycan compositions were identified in total (FIG. 4K). As shown in FIG. 4E, mono-, di-, and trifucosylated glycans were detected in single HeLa cells following LPS treatment, while N-glycan profiling of single HeLa cells before LPS treatment resulted only in the assignment of mono- and difucosylated glycans. LPS activation of HeLa cells also resulted in the detection of penta- and hexasialylated glycans, undetectable in single HeLa cells before LPS treatment (FIG. 4F). Overall, the fractional distributions of N-glycans detected in single HeLa cells were significantly different before and after LPS treatment. Mono-, di-, and trifucosylated N-glycans accounted for 39%, 13%, and 4%, respectively, in LPS-treated HeLa cells (vs. 51%, 3%, and 0%, respectively, in untreated HeLa cells, FIG. 4E). Mono-, di-, tri-, tetra-, penta-, and hexasialylated N-glycans accounted for 13%, 36%, 11%, 10%, 2%, and 1%, respectively, in LPS-treated HeLa cells (vs. 4%, 34%, 23%, 22%, 0%, and 0%, respectively, in untreated HeLa cells, FIG. 4F). As shown in FIG. 10A, 34% of the N-glycans detected in LPS-treated HeLa cells were also identified in untreated HeLa cells. Interestingly, 50 N-glycans were uniquely detected in LPS-treated HeLa cells, among which 44% were neutral (including trifucosylated) glycans, and 56% were sialylated (including penta- and hexasialylated) glycans. A quantitative comparison of glycan abundances was also conducted. As shown in FIGS. 6E and 10C, the total abundances of fucosylated glycans detected in single HeLa cells were not significantly different (p=0.6) before and after LPS treatment, and accounted for 75% and 78%, respectively, based on peak intensity measurements. However, the stimulation of HeLa cells with LPS resulted in significantly altered sialylation profiles. As shown in FIG. 6F, a high increase in the total abundance of HeLa cell-derived nonsialylated glycans was noticed (p=0.06) after LPS treatment. In addition, the total abundances of mono-, tri-, and tetrasialylated glycans were significantly different (p<0.05) in untreated and LPS-treated HeLa cells (FIG. 10D). These results confirmed that LPS stimulation of HeLa cells induced significant changes in HeLa cell N-glycome profiles, which could be detected at the single-cell level using the present method.

Significant alterations of U87 cell N-glycome profiles were also observed at the single-cell level when U87 cells were treated with LPS, compared to the untreated U87 cells. CE-MS analysis of single U87 cells after LPS treatment resulted in the detection of 55±30 N-glycans per single U87 cell (n=5), and in the assignment of 161 non-redundant N-glycan compositions in total. 68% of the N-glycans identified in LPS-treated U87 cells were fucosylated, and the fractional distributions were 36%, 15%, 11%, 1%, 1%, and 4% for mono-, di-, tri-, tetra-, penta-, and hexafucosylated N-glycans, respectively (FIG. 4G). Mono-, di-, tri-, tetra-, penta-, hexa-, and heavily (i.e., ≥7 SiA residues) sialylated N-glycans accounted for 12%, 22%, 18%, 13%, 2%, 0%, and 1%, respectively (FIG. 4H). As shown in FIG. 10B, 92 glycans were commonly detected in LPS-treated and untreated U87 cells. Yet, 69 glycans were uniquely detected in LPS-treated U87 cells, among which many pentasialylated and several heavily sialylated N-glycans containing up to 13 SiA residues. These results indicate that LPS treatment induced a modification in the type and structure of biosynthesized sialylated glycans in U87 cells. These unique, highly sialylated glycans detected on the surface of LPS-stimulated U87 cells at the single-cell level using the method described herein would be challenging to detect using alternative total cellular glycomic analysis or lectin-based methodologies. Indeed, based on results presented herein, the fractional distributions (FIGS. 4G-4H) and fractional abundances (FIGS. 6F and 10F) of sialylated N-glycans identified in single U87 cells did not change significantly before and after LPS treatment, and only a thorough qualitative and quantitative comparison of released single-cell surface N-glycans could reveal such sialylation subtlety. In addition, the comparative quantitative analysis of fucosylated glycans detected in single U87 cells showed that their total abundances significantly decreased from 74% to 54% (p<0.05) when U87 cells were treated with LPS, based on peak intensity measurements (FIGS. 6E and 10E). These results clearly demonstrate that LPS treatment induced a downregulation of fucosylated glycans in U87 cells that can be detected at the single-cell level.

FIG. 6C depicts the results of Euclidean distance-based hierarchical clustering of quantitative glycomic profiles of 84 representative N-glycans detected in LPS-treated and untreated HeLa and U87 single cells (i.e., the set of 47 glycans highly representative of HeLa and U87 cells, described above, was extended with 37 representative glycans detected in LPS-treated HeLa and LPS-treated U87 cells, respectively, see Methods section). This clustering analysis resulted in a plausible differentiation of two clades, corresponding to treated/untreated HeLa cells, and treated/untreated U87 cells, respectively. In addition, four repetitive analyses of LPS-treated HeLa cells and four repetitive analyses of LPS-treated U87 cells were clustered together. These results further demonstrated the uniqueness of HeLa and U87 cell line N-glycomes, and showed that both cell lines exhibited different biological responses to LPS treatment. PCA was also conducted on the datasets generated from the analyses of LPS-treated and untreated HeLa and U87 cells, selecting 57 representative glycans (FIG. 6D). As shown in FIG. 6D, four distinct clusters corresponding to LPS-treated and untreated HeLa cells, and LPS-treated and untreated U87 cells, respectively, were differentiated and confirmed a pronounced effect of LPS treatment. As expected, a partial overlap was observed between the two single HeLa cell-related clusters and the two single U87 cell-related clusters. Overall, these results indicated the undeniable biological effect of LPS stimulation on HeLa and U87 cell N-glycomes, which could be detected at the single-cell level using our developed SCG workflow.

The present technology offers several novel and unusual features. The method enables straightforward, unbiased, accurate, and deep qualitative and quantitative glycomic profiling of single mammalian cells. The method is based on innovative in-capillary sample processing coupled online to ultra-high sensitivity label-free CE-MS analysis of released glycans. The method also enables deep and highly informative glycan profiling of sub-0.5 ng-levels of model proteins and nL to pL levels of plasma volume equivalents (e.g., 5 pL of total plasma can be loaded in the CE capillary for straightforward glycan profiling). The numbers of N-glycans identified in the four types of analyzed blood-derived isolates (IgM, IgG, total plasma, and EVs) described here exceed by ˜7-fold those reported in other N-glycan profiling studies of similar complexity blood-derived isolates. Further, optimization of ionization, desolvation, and CE-MS conditions achieved the highest sensitivity levels available for glycan analysis. Further, even highly fucosylated (5-7 fucose residues) and highly sialylated (5-14 salic acid residues) N-glycans, which are difficult to detect using the prior glycan labeling-based methodologies, can be detected and structurally characterized using the present label-free CE-MS-based workflow. Such peculiar N-glycans can now be included in biopharmaceutical and clinical research and development efforts as new classes of targets. Moreover, unmatched separation performance of the present technology was achieved with the present CE-MS method, which allows separation and analysis of positional and linkage glycan isomers in a single CE-MS analysis. Such isomeric differentiation is a challenge for other separation approaches.

The present technology has several advantages over related technology of the prior art. (i) The developed technique offers a vastly simplified, in-capillary sample preparation that eliminates the sample losses associated with sample handling and transfer steps in off-line approaches, and it allows straightforward and in-depth glycan profiling of extremely small amounts of sample, such as nL or pL equivalent amounts of blood isolates as well as small populations of cells or even single cells. Up to now, there are no established methods for single-cell glycome analysis due to the inability to amplify glycan sequences and sample losses associated with any sample processing and glycan labeling. To date, only a few lectin-based glycan profiling technologies have been developed for single-cell glycomics. These approaches involve tedious, expensive, and time-consuming analytical workflows with sophisticated instrumentation and do not allow the direct analysis, quantitation, and accurate structural characterization of the glycans. Furthermore, some cell surface glycans may not interact with the lectins selected in the developed methodologies. (ii) The present glycan profiling technique is quick, requiring about 3 hours per single-cell or blood isolate analysis, including sample loading, in-capillary glycan release, and CE-MS analysis. It allows a well-controlled injection of individual cells, and requires only affordable analytical instruments. (iii) N-glycans are analyzed in their native underivatized state, preserving their endogenous glycan features and eliminating the drawbacks associated with labeling procedures, including incomplete derivatization, over-labeling, formation of side-products, sample losses during sample labeling and cleanup steps, ionization suppression and MS signal interference by the labeling reagent, and high levels of defucosylation/desialylation during sample preparation and MS analysis. With the present label-free approach higher levels of sialylation and fucosylation are detected because of lower levels of electrospray ionization and source-induced decay during MS analysis. (iv) The present method allows increased depth of glycan profiling, yielding higher numbers and varieties of glycans. (v) The high sensitivity and high dynamic range of detection (over 5 orders of magnitude) of the present technique enables the detection of highly fucosylated (5-7 fucose residues) and highly/heavily sialylated (5-14 salic acid residues) N-glycans, most of which were not reported before.

Uses of the present methodology include glycan profiling of small populations of cells (≤20 cells) and single cells, extracellular vesicles and single microvesicles, and limited amounts of biological or clinical samples, such as minimally-invasive liquid microbiopsies, and tissue isolates. The methods also can be used for discovery of novel diagnostic, prognostic, and treatment-monitoring of specific disease biomarkers based on glycosylation profile alterations associated with diseases and medical conditions. The present methods also can be integrated into development of a vast single-cell glycomics or multi-omics platform, which could provide crucial information on biological mechanisms underlying complex diseases, unachievable by merging data sets obtained from mono-omics studies of different cells. Characterization of glycosylation levels in biopharmaceuticals (e.g., therapeutic proteins and vaccines) can be obtained using the present technology.

### EXAMPLES

### Example 1. Materials and General Methodology

### Materials and Chemicals

Deionized water, methanol (99.9%, LC/MS Grade), Gibco F12-K medium, Gibco DMEM medium, Gibco 0.25% Trypsin-EDTA, Gibco penicillin-streptomycin (P/S), phosphate-buffered saline (PBS), CellMask™ plasma membrane stain (green), LIVE/DEAD™ fixable green dead cell stain kit, and lipopolysaccharide (LPS) were obtained from Thermo Fisher Scientific (Waltham, MA). 1 N NaOH, 1 N HCl, 5 N ammonium hydroxide, glacial acetic acid (99.99%), ultra-high purity ammonium acetate (99.999%), trypan blue, and total human serum IgM and human serum IgG isolates (purity ≥95%, based on non-reduced SDS-PAGE and verified by nanoLC-MS/MS of tryptic digests) were purchased from Sigma-Aldrich (St. Louis, MO). PNGase F enzyme was from New England Biolabs (Ipswich, MA). Fetal bovine serum (FBS) was purchased from R&D Systems (Minneapolis, MN). Platelet-free anticoagulated with EDTA pooled total human plasma (from blood donated by self-declared healthy male donors of 23-67 years old) was kindly provided by Prof. Ghiran's laboratory (BIDMC, Boston, MA). All bare fused silica (BFS) capillaries (91 cm×30 μm i.d.×150 μm o.d.) with sheathless CESI-MS emitters in OptiMS™ cartridges were from SCIEX (Redwood City, CA). Aquapel® was purchased at Pittsburgh Glass Works (Pittsburgh, PA).

### Cell Culture

HeLa-S3 and U87-MG cell lines (called HeLa and U87 cells thereafter) were from ATCC (Manassas, VA). HeLa cells were cultured in suspension at 37° C. in a complete F-12K medium supplemented with 10% FBS, 1% P/S, and 5% CO₂. The cell density was maintained within a range of 2×10⁵-1×10⁶ cells/mL. Adherent U87 cells were cultured at 37° C. in DMEM medium supplemented with 10% FBS, 1% P/S, and 5% CO₂. Upon reaching confluence, one flask of U87 cells was split into five flasks. Cells were stained with trypan blue and counted using a 2-chip disposable hemocytometer (Bulldog Bio, Portsmouth, NH) to estimate the cell density and viability.

### LPS Treatment of HeLa and U87 Cells

2 or 4 μL of 2.5 mg/mL LPS were added to the 5 or 10 mL culture media in each HeLa or U87 cell culture flask, to get a final LPS concentration of 1 pg/mL. The HeLa and U87 cells were exposed to LPS for 24 h before being harvested and analyzed.

### Cell Pellet Collection

HeLa and U87 cells were collected, washed, counted, and subsequently centrifuged into pellets prior to CE-MS analysis. The HeLa cell pellets were obtained by direct centrifugation of the HeLa cell culture suspension at 300×g for 5 min. To detach the U87 cells from the flask bottom, 0.25% trypsin-EDTA was added, followed by incubation at 37° C. for 5 min. The digestion was stopped by adding complete DMEM medium, and the detached U87 cells were centrifuged at 300×g for 5 min to obtain the cell pellets. HeLa and U87 cell pellets were washed three times with 1×PBS, and their viability and density were assessed using a 2-chip disposable hemocytometer prior to the final centrifugation at 300×g for 5 min. The cell viability was typically >90%. The cell pellets were kept on ice until their use.

Offline Cell Loading into the CE Capillary

One or five cells were loaded offline into the silica surface OptiMS Cartridge, following the protocol described in our previous work³², with modifications. The cell loading process was visualized and monitored under an IX83 microscope (Olympus, Center Valley, PA), using a 10× magnification. First, the inlet of the CE capillary separation line was immobilized on a glass slide (pretreated with Aquapel®) placed under the microscope. Then, the capillary inlet was immersed in a 40 μL droplet of 1 mM ammonium acetate pH 6.7. A hydrodynamic flow was generated by manually lifting or lowering by ˜45 cm the electrospray emitter tip of the capillary (separation line outlet) to generate an ultra-low flow rate of ˜140 pL/s and enable precise control of the cell influx. Flow towards the separation line inlet was created by lifting the emitter tip to expel air bubbles before cell loading or dislodge unwanted cells after cell loading. For cell loading, 5 μL of a cell suspension at ˜5 cells/nL was mixed with the droplet in which the separation line inlet was immersed, while the emitter tip of the capillary was held at the same height as the separation line inlet to prevent any forward or backward flow. The cell-containing droplet was gently agitated with a pipet tip until a target single cell (e.g., with the desired size and morphology) was observed in close proximity to the inlet. Then, the emitter tip of the capillary was lowered to introduce the cell into the capillary, and the flow was maintained until the cell was located approximately 500 μm away from the capillary inlet for targeted cell injection. The same procedure was repeated several times to load manually the desired number of cells.

### Cell Staining and Visualization

To record the cell morphology and size distribution, 5 μL of a suspension of unstained cells in 1×PBS (with a cell density of ˜1×10⁶ cells/mL) were deposited on a glass slide and imaged with the microscope under bright field at 10× magnification. For improved visualization of the cell morphology and membrane integrity, the cells were stained with CellMask™ plasma membrane green stain, following a procedure adapted from the manufacturer's protocol. The 1,000× concentrated stain solution was diluted to 1× working solution with PBS. Subsequently, the cell pellet was resuspended with the working solution to an approximate cell density of 1×10⁶ cells/mL. Then, the cells were incubated in the dark for 30 min, followed by three washes with PBS to remove the excess stain. For fluorescence microscopy imaging of stained cells loaded within the capillary, the polyimide coating was removed before the experiments to avoid interference. To determine the cell viability and membrane integrity under the selected in-capillary sample processing conditions (i.e., after 60 min of incubation with the PNGase F enzyme in 1 mM ammonium acetate pH 6.7 buffer), the cells were stained with LIVE/DEAD™ fixable green dead cell stain. For this, one vial of the fluorescent dye was resuspended with 50 μL of DMSO. HeLa cells were harvested, washed, and resuspended with 1 mM ammonium acetate pH 6.7 to an approximate cell density of 5×10⁵ cells/mL. Then, 1 μL of the resuspended dye was added to 1 mL of the cell suspension. Finally, 20 μL of the stained cells were mixed with 15 mlU of PNGase F. Bright field and fluorescence-based microscopic images were acquired at different time points to evaluate the cell viability and morphology during the deglycosylation step with PNGase F.

### Preparation and Characterization of EV Isolate

Plasma-derived EVs were isolated using a size exclusion chromatography (SEC) column with a Sepharose CL-2B stationary phase. Briefly, 100 μL of platelet-free anticoagulated with EDTA pooled human plasma (from blood donated by self-declared healthy male donors of 23-67 years old) were loaded on the SEC column. EVs were eluted from the SEC column with 0.1×dPBS, and the EV-containing fractions were pooled. The pooled EV fractions were then concentrated using Amicon® 30 kDa MWCO ultrafiltration devices to a final volume of ˜33 μL, and stored at 4° C. until their analysis. The approximate EV particle concentration was estimated to be 1×10¹⁰ EV particles/mL, based on a combination of EV counting, using tunable resistive pulse sensing (TRPS), nano-flow cytometry, and immunoaffinity-based interferometry.

### MS Instrumentation and Techniques

The CE capillary was interfaced with an Orbitrap™ Fusion Lumos™ mass spectrometer using a Nanospray Flex ion source (both Thermo Fisher Scientific, Bremen, Germany). All analyses were carried out in negative ESI mode. The nanoelectrospray potential was set to ˜1.8 kV. The ion transfer tube (ITT) temperature was set to 150° C. (the distance between the electrospray emitter tip and the MS ITT was set to ˜5 mm). The CE-MS analyses were performed with automatic gain control (AGC) of 1×10⁶ or 250%, a maximum injection time of 250 ms, 5 microscans, an S-lens voltage set to 65 eV, the nominal resolving power of 120,000 at 200 m/z, and in-source collision-induced dissociation (ISCID) at 70 eV. For CE-MS² experiments, instrument resolving power was set at 60,000 at 200 m/z with 1 microscan. AGC was set to 2×10⁵ with a maximum injection time of 1,000 ms. An isolation window of 2 m/z was selected, and 32 eV was determined to provide the optimum normalized collision energy.

### Data Analysis

For data acquisition and processing, Xcalibur™ (v. 3.1) software was used. CE-MS data were processed with GlycReSoft (v. 3.10) software (Boston University, Boston, MA, USA). Analyses of CE-MS² data were performed with SimGlycan (v. 5.91) software (Premier Biosoft, Palo Alto, CA, USA). The generated results were based on the processing of three replicate (model proteins, total plasma, and EVs) and five repetitive (mammalian cells) analyses. For CE-MS¹ processing with GlycReSoft, a mass matching error tolerance of 20 ppm was used in all searches. Up to six charge states, and sodium and ammonium adducts were included in the search. Other parameters were the same as described in our previous reports^{40,41}. The glycan identification analysis of the CE-MS data was conducted using database searches against in-house built mammalian database (version of December 2020) encompassing 27,335 N-glycan compositions (the mammalian database provided with the GlycReSoft software package encompasses 1,766 N-glycan compositions). For CE-MS² processing with SimGlycan, a 20 ppm precursor mass tolerance and a 10 ppm fragment mass tolerance were used in all searches. Non-labeled glycans (unmodified or with sodium adduction) were searched selecting the options “Underivatized” and “Free” in the chemical derivatization and reducing terminal windows, respectively. Other parameters were as described in our previous studies^{40,41}. The glycan composition identification results were mainly based on CE-MS data processing using GlycReSoft. As additional verification of the plausible glycan identifications made using GlycReSoft, several supplementary levels of manual data examination were applied according to our recent studies^{40,41}. In brief, this verification included 1. Predictable trends in CE-MS migration patterns, 2. Charge state and isotopic distributions characteristic to glycan ions, 3. Detection of neutral losses, and 4. Manual examination of CE-MS² data for low intensity parent ions. The relative quantitation of the detected N-glycans was based on the single-stage MS signal intensities or peak areas of the detected N-glycans that were normalized with respect to the summed MS signal intensities or peak areas of all the N-glycans detected in the sample. In addition, a qualitative comparison was performed based on the fractional distributions corresponding to the number of specific species (e.g., diasialylated glycans) out of the total number of N-glycans detected and identified in the analyzed biological specimens.

The bar charts with individual data points, mean values, and error bars were plotted using the R language and ggplot2 package in the rStudio development environment (2023.03.0+386 “Cherry Blossom” Release). The R language in the rStudio development environment was also used to perform statistical ANOVA and paired t-tests. The open-access tBtools-II (v1.120) software⁴² was employed to generate heatmap clustering, utilizing the Euclidean distance-based clustering method and the complete cluster approach. For data clustering, N-glycan abundances (based on peak intensities) were normalized with respect to the summed abundances of all the N-glycans detected in the analyzed biological sample, and the clustering was done using the normalized abundance values after imputing 10% of minimum abundance for missing values followed by log₂ transformation. The PCA plots were created with the open-access version of ClustVis (https://biit.cs.ut.ee/clustvis/software⁴³. The average cell diameters of HeLa and U87 cells were measured using the open-access ImageJ (v1.53k) software. The glycan structures were designed with the open-access version of GlycoWorkBench (v2.0). Other schematic images (e.g., cell structure illustration) were built with the BioRender graphical tool.

For the Euclidean distance-based hierarchical clustering of single HeLa and single U87 cells before LPS treatment, glycans that were detected in at least three CE-MS analyses out of the ten total repetitive analyses (i.e., five repetitive analyses for single HeLa cells and five repetitive analyses for single U87 cells) were selected. This selection generated a set of 47 glycans highly representative of single HeLa and single U87 cells. For the Euclidean distance-based hierarchical clustering of single HeLa and single U87 cells after LPS treatment, glycans that were detected in at least two CE-MS analyses out of the five repetitive analyses of LPS-treated HeLa cells, and glycans that were detected in at least two CE-MS analyses out of the five repetitive analyses of LPS-treated U87 cells were selected and added to the above-described 47 glycans that are highly representative of HeLa and U87 untreated cells. For the PCA analysis of the glycans identified in single HeLa and single U87 cells after LPS treatment, more stringent parameters were used. In this case, glycans that were detected in at least three CE-MS analyses out of the five repetitive analyses of LPS-treated HeLa cells, and glycans that were detected in at least three CE-MS analyses out of the five repetitive analyses of LPS-treated U87 cells were selected and added to the above-described 47 glycans highly representative of HeLa and U87 untreated cells.

### Example 2. Sample Loading, In-Capillary Processing, and CE-MS Analysis

### Model Glycoproteins

IgM and IgG were isolated from blood serum by size-exclusion chromatography (IgM) and ion-exchange chromatography (IgG), sample injections were performed at 1 or 5 psi for 6 s, corresponding to 1 and 5 nL injection volumes, respectively (i.e., 0.16 and 0.8% of the capillary volume, respectively). Three replicate analyses were performed with the injection of 0.1 ng and 5 ng of IgM, corresponding to ˜60 pL and ˜3,000 pL of human serum, respectively. Three replicate analyses were performed with the injection of 0.5 ng and 5 ng of IgG, corresponding to ˜50 pL and ˜500 pL of human serum, respectively.

### Total Plasma Isolate

1 mL of whole blood plasma isolate was centrifuged at 16,000×g for 20 min at 4° C., and the supernatant was carefully pipetted to avoid collecting the lipid layer. For CE-MS analysis of total plasma, sample injections were performed at 1 psi for 6 s, corresponding to 1 nL injection volumes, and 5, 50, or 500 pL of plasma, depending on the dilution of the “pre-processed” plasma sample in water.

### EV Isolate

Sample injections were performed at 1 psi for 6 s (1 nL injected) or 5 psi for 60 s (50 nL injected), corresponding to injected amounts equivalent to ˜3 nL and ˜150 nL of plasma, respectively.

### Cell Analysis

Cell pellets were resuspended in 200 μL of 1 mM ammonium acetate pH 6.7 to get a final cell density of ˜5 cells/nL. For online cell loading of ˜10 cells, 2 nL of a cell suspension at ˜5 cells/nL were injected, applying 1 psi for 12 s. In the offline cell loading mode, 1 to 5 cells were selected and injected manually as described above, and the 1-5 cell-containing plugs corresponded to ˜4-6 nL injection volumes, based on microscope visualization. Owing to cell size variations, sets of five repetitive analyses were systematically performed with one (offline injection), five (offline injection), and ˜ten (online “bulk sample” injection) mammalian cells for each cell type (HeLa and U87 cells). CE-MS analyses of a blank sample of water were performed systematically to confirm insignificant levels of carryover derived from the analysis of preceding biological samples (blood-derived isolates and mammalian cells). For single-cell analysis, a water blank sample was analyzed between each single-cell injection. CE-MS analyses of the cell suspension medium (i.e., 1 mM ammonium acetate cell suspension buffer) were also performed. For these control analyses, 2-4 nL of water or cell suspension medium were injected inside the capillary and processed using the developed workflow, including the digestion step with PNGase F.

Off-Line Cell Loading into the CE Capillary

One to five cells were loaded off-line into the silica surface OptiMS Cartridge. The cell loading process was visualized and monitored under an IX83 microscope (Olympus, Center Valley, PA), using a 10× magnification. First, the inlet of the CE capillary separation line was immobilized on a glass slide (pretreated with Aquapel®) placed under the microscope. Then, the capillary inlet was immersed in a 40 μL droplet of 1 mM ammonium acetate pH 6.7. A hydrodynamic flow was generated by manually lifting or lowering by ˜45 cm the electrospray emitter tip of the capillary (separation line outlet) to generate an ultra-low flow rate of ˜140 pL/s and enable precise control of the cell influx. Flow towards the separation line inlet was created by lifting the emitter tip to expel air bubbles before cell loading or dislodge unwanted cells after cell loading. For cell loading, 5 μL of a cell suspension at ˜5 cell/nL was mixed with the droplet in which the separation line inlet was immersed, while the emitter tip of the capillary was held at the same height as the separation line inlet to prevent any forward or backward flow. The cell-containing droplet was agitated with a pipet tip until a single cell with the desired size and morphology was close to the inlet. Then, the emitter tip of the capillary was lowered to introduce the cell into the capillary and the flow was maintained until the cell was located approximately 500 μm away from the capillary inlet to prevent the cell dislodging. The same procedure was repeated several times to load manually the desired number of cells.

### In-Capillary Sample Processing and CE Methods

In-capillary sample processing for N-glycan release with PNGase F and CE-MS experiments were conducted using a CESI 8000™ instrument (SCIEX). In all experiments, bare fused silica (BFS) OptiMS capillaries (91 cm×30 μm i.d.×150 μm o.d.) were used. Prior to each online or offline sample injection, a series of rinses of the separation and conductive lines were performed. For the separation capillary, these rinses included: MeOH (100 psi, 10 min), 0.1 M NaOH (100 psi, 3 min), 0.1 M HCl (100 psi, 3 min), and Milli-Q water (100 psi, 5 min), followed by the background electrolyte (BGE) (100 psi, 7 min). The conductive line was rinsed with the BGE (100 psi, 2 min). Before and after online (model glycoproteins, whole plasma, EVs, and ˜ten cells (referred to as “bulk cells” in this study)) or offline (1-5 cells) sample loading into the CE capillary inlet, a plug (1 or 2 nL applying 1 psi for 6 or 12 s) of a PNGase F digestion solution at 1.1 mlU/pL in 7 mM NaCl, 3 mM Tris-HCl, and 0.7 mM Na₂EDTA was injected into the capillary using the CESI 8000 instrument. For offline cell loading, the CE cartridge was removed from the CESI instrument after the injection of the first PNGase F plug for manual cell loading, as described above, and placed back in the CESI instrument for subsequent in-capillary sample processing. After online or offline sample loading, a short plug of water (1 nL) was injected before the injection of the second PNGase F plug, followed by a short plug (0.5 or 1 nL) of 50 mM ammonium acetate pH 6.7. Then, two voltage pulses of 20 kV were applied in normal and reverse polarity for 30 s with the BGE composed of 10 mM (ionic strength) ammonium acetate pH 4.5 with 10% isopropanol, before incubating the capillary inlet in a vial containing 50 mM ammonium acetate pH 6.7 for either 30 min (model glycoproteins, whole plasma, and EVs) or 60 min (mammalian cells). After the in-capillary incubation step (performed at −12° C.) for N-glycan release with PNGase F, a BGE plug (10 psi for 10 s (model glycoproteins and whole plasma) or 10 psi for 60 s (mammalian cells)) was injected in the capillary prior to label-free CE-MS analysis of released N-glycans performed as described below. All CE methods employed 20 kV in reverse polarity with a voltage ramp time of 1 min. The CE-MS experiments were carried out with a BGE of 10 mM (ionic strength) ammonium acetate pH 4.5 with 10% isopropanol. This BGE generated a relatively low cathodic EOF (μ_{EOF} 2.02×10⁻⁸ m²/V/s) based on the detection of a neutral marker (acetaminophen). All CE-MS analyses were performed with a CE supplemental pressure (SP) of 5 psi, which was applied 18 min after switching on the CE voltage at the beginning of the CE run. Due to the variability in the manually injected cell plugs (performed offline), the migration time ranges in CE-MS analysis of mammalian cells were normalized, based on the most abundant detected glycan species.

### MS Instrumentation and Techniques

The CE capillary was interfaced with an Orbitrap™ Fusion Lumos™ mass spectrometer using a Nanospray Flex ion source (both Thermo Fisher Scientific, Bremen, Germany). All analyses were carried out in negative ESI mode. The nanoelectrospray potential was set to ˜1.8 kV. The ion transfer tube (ITT) temperature was set to 150° C. (the distance between the electrospray emitter tip and the MS ITT was set to 5 mm). The CE-MS analyses were performed with automatic gain control (AGC) of 1×106 or 250%, a maximum injection time of 250 ms, 5 microscans, a S-lens voltage set to 65 eV, the nominal resolving power of 120,000 at 200 m/z, and in-source collision-induced dissociation (ISCID) at 70 eV. For CE-MS2 experiments, instrument resolving power was set at 60,000 at 200 m/z with 1 microscan. AGC was set to 2×105 with a maximum injection time of 1,000 ms. An isolation window of 2 m/z was selected, and 32 eV was determined to provide the optimum normalized collision energy. CE-MS1 was performed as described above.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1 Varki, A. Essentials in Glycobiology, 4th Edition. Cold Spring Harbor (NY) (2022).
2 Ohtsubo, K. & Marth, J. D. Glycosylation in cellular mechanisms of health and disease. Cell 126, 855-867, doi:10.1016/j.cell.2006.08.019 (2006).
3 Wolfert, M. A. & Boons, G. J. Adaptive immune activation: glycosylation does matter. Nat Chem Biol 9, 776-784, doi:10.1038/nchembio.1403 (2013).
4 Mockl, L. The Emerging Role of the Mammalian Glycocalyx in Functional Membrane Organization and Immune System Regulation. Front Cell Dev Biol 8, 253, doi:10.3389/fcell.2020.00253 (2020).
5 Reily, C., Stewart, T. J., Renfrow, M. B. & Novak, J. Glycosylation in health and disease. Nat Rev Nephrol 15, 346-366, doi:10.1038/s41581-019-0129-4 (2019).
6 Edgar, L. J. Engineering the Sialome. ACS Chem Biol 16, 1829-1840, doi:10.1021/acschembio.1c00273 (2021).
7 Gao, Y., Luan, X., Melamed, J. & Brockhausen, I. Role of Glycans on Key Cell Surface Receptors That Regulate Cell Proliferation and Cell Death. Cells 10, doi:10.3390/cells10051252 (2021).
8 Christiansen, M. N. et al. Cell surface protein glycosylation in cancer. Proteomics 14, 525-546, doi:10.1002/pmic.201300387 (2014).
9 Thomas, D., Rathinavel, A. K. & Radhakrishnan, P. Altered glycosylation in cancer: A promising target for biomarkers and therapeutics. Biochim Biophys Acta Rev Cancer 1875, 188464, doi:10.1016/j.bbcan.2020.188464 (2021).
10 Lumibao, J. C., Tremblay, J. R., Hsu, J. & Engle, D. D. Altered glycosylation in pancreatic cancer and beyond. J Exp Med 219, doi:10.1084/jem.20211505 (2022).
11 Costa, A. F., Campos, D., Reis, C. A. & Gomes, C. Targeting Glycosylation: A New Road for Cancer Drug Discovery. Trends Cancer 6, 757-766, doi:10.1016/j.trecan.2020.04.002 (2020).
12 Caval, T., Alisson-Silva, F. & Schwarz, F. Roles of glycosylation at the cancer cell surface: opportunities for large scale glycoproteomics. Theranostics 13, 2605-2615, doi:10.7150/thno.81760 (2023).
13 Haukedal, H. & Freude, K. K. Implications of Glycosylation in Alzheimer's Disease. Front Neurosci 14, 625348, doi:10.3389/fnins.2020.625348 (2020).
14 Schedin-Weiss, S. et al. Glycan biomarkers for Alzheimer disease correlate with T-tau and P-tau in cerebrospinal fluid in subjective cognitive impairment. FEBS J 287, 3221-3234, doi:10.1111/febs.15197 (2020).
15 Smith, B. A. H. & Bertozzi, C. R. The clinical impact of glycobiology: targeting selectins, Siglecs and mammalian glycans. Nat Rev Drug Discov 20, 217-243, doi:10.1038/s41573-020-00093-1 (2021).
16 Berois, N., Pittini, A. & Osinaga, E. Targeting Tumor Glycans for Cancer Therapy: Successes, Limitations, and Perspectives. Cancers (Basel) 14, doi:10.3390/cancers14030645 (2022).
17 Lei, Y. et al. Applications of single-cell sequencing in cancer research: progress and perspectives. J Hematol Oncol 14, 91, doi:10.1186/s13045-021-01105-2 (2021).
18 Tajik, M., Baharfar, M. & Donald, W A. Single-cell mass spectrometry. Trends Biotechnol 40, 1374-1392, doi:10.1016/j.tibtech.2022.04.004 (2022).
19 Wu, A. R. et al. Quantitative assessment of single-cell RNA-sequencing methods. Nat Methods 11, 41-46, doi:10.1038/nmeth.2694 (2014).
20 Misevic, G. Single-cell omics analyses with single molecular detection: challenges and perspectives. J Biomed Res 35, 264-276, doi:10.7555/JBR.35.20210026 (2021).
21 Kunej, T. Rise of Systems Glycobiology and Personalized Glycomedicine: Why and How to Integrate Glycomics with Multiomics Science? OMICS 23, 615-622, doi:10.1089/omi.2019.0149 (2019).
22 Vistain, L. F. & Tay, S. Single-Cell Proteomics. Trends Biochem Sci 46, 661-672, doi:10.1016/j.tibs.2021.01.013 (2021).
23 Kelly, R. T. Single-cell Proteomics: Progress and Prospects. Mol Cell Proteomics 19, 1739-1748, doi:10.1074/mcp.R120.002234 (2020).
24 Mund, A., Brunner, A. D. & Mann, M. Unbiased spatial proteomics with single-cell resolution in tissues. Mol Cell 82, 2335-2349, doi:10.1016/j.molcel.2022.05.022 (2022).
25 Cong, Y et al. Ultrasensitive single-cell proteomics workflow identifies >1000 protein groups per mammalian cell. Chem Sci 12, 1001-1006, doi:10.1039/d0sc03636f (2020).
26 Budnik, B., Levy, E., Harmange, G. & Slavov, N. SCoPE-MS: mass spectrometry of single mammalian cells quantifies proteome heterogeneity during cell differentiation. Genome Biol 19, 161, doi:10.1186/s13059-018-1547-5 (2018).
27 Leduc, A., Huffman, R. G., Cantlon, J., Khan, S. & Slavov, N. Exploring functional protein covariation across single cells using nPOP. Genome Biol 23, 261, doi:10.1186/s13059-022-02817-5 (2022).
28 Cong, Y. et al. Improved Single-Cell Proteome Coverage Using Narrow-Bore Packed NanoLC Columns and Ultrasensitive Mass Spectrometry. Anal Chem 92, 2665-2671, doi:10.1021/acs.analchem.9b04631 (2020).
29 Zhu, Y. et al. Nanodroplet processing platform for deep and quantitative proteome profiling of 10-100 mammalian cells. Nat Commun 9, 882, doi:10.1038/s41467-018-03367-w (2018).
30 Matzinger, M., Muller, E., Durnberger, G., Pichler, P. & Mechtler, K. Robust and Easy-to-Use One-Pot Workflow for Label-Free Single-Cell Proteomics. Anal Chem 95, 4435-4445, doi:10.1021/acs.analchem.2c05022 (2023).
31 Gebreyesus, S. T. et al. Streamlined single-cell proteomics by an integrated microfluidic chip and data-independent acquisition mass spectrometry. Nat Commun 13, 37, doi:10.1038/s41467-021-27778-4 (2022).
32 Johnson, K. R., Gao, Y., Gregus, M. & Ivanov, A. R. On-capillary Cell Lysis Enables Top-down Proteomic Analysis of Single Mammalian Cells by CE-MS/MS. Anal Chem 94, 14358-14367, doi:10.1021/acs.analchem.2c03045 (2022).
33 Vaclavek, T. & Foret, F. Microfluidic device integrating single-cell extraction and electrical lysis for mass spectrometry detection of intracellular compounds. Electrophoresis 44, 313-322, doi:10.1002/elps.202100379 (2023).
34 Fujitani, N. et al. Total cellular glycomics allows characterizing cells and streamlining the discovery process for cellular biomarkers. Proc Natl Acad Sci USA 110, 2105-2110, doi:10.1073/pnas.1214233110 (2013).
35 Homan, K. et al. Alteration of the Total Cellular Glycome during Late Differentiation of Chondrocytes. Int J Mol Sci 20, doi:10.3390/ijms20143546 (2019).
36 Kearney, C. J. et al. SUGAR-seq enables simultaneous detection of glycans, epitopes, and the transcriptome in single cells. Sci Adv 7, doi:10.1126/sciadv.abe3610 (2021).
37 Minoshima, F., Ozaki, H., Odaka, H. & Tateno, H. Integrated analysis of glycan and RNA in single cells. iScience 24, 102882, doi:10.1016/j.isci.2021.102882 (2021).
38 Ma, T. et al. Single-cell glycomics analysis by CyTOF-Lec reveals glycan features defining cells differentially susceptible to HIV. Elife 11, doi:10.7554/eLife.78870 (2022).
39 Dworkin, L. A., Clausen, H. & Joshi, H. J. Applying transcriptomics to study glycosylation at the cell type level. Iscience 25, doi:ARTN 10441910.1016/j.isci.2022.104419 (2022).
40 Marie, A. L., Ray, S. & Ivanov, A. R. Highly-sensitive label-free deep profiling of N-glycans released from biomedically-relevant samples. Nat Commun 14, 1618, doi:10.1038/s41467-023-37365-4 (2023).
41 Marie, A. L. et al. High-Sensitivity Glycan Profiling of Blood-Derived Immunoglobulin G, Plasma, and Extracellular Vesicle Isolates with Capillary Zone Electrophoresis-Mass Spectrometry. Anal Chem 93, 1991-2002, doi:10.1021/acs.analchem.0c03102 (2021).
42 Chen, C. et al. TBtools: An Integrative Toolkit Developed for Interactive Analyses of Big Biological Data. Mol Plant 13, 1194-1202, doi:10.1016/j.molp.2020.06.009 (2020).
43 Metsalu, T. & Vilo, J. ClustVis: a web tool for visualizing clustering of multivariate data using Principal Component Analysis and heatmap. Nucleic Acids Res 43, W566-570, doi:10.1093/nar/gkv468 (2015).
44 Lloyd, K. A., Wang, J., Urban, B. C., Czajkowsky, D. M. & Pleass, R. J. Glycan-independent binding and internalization of human IgM to FCMR, its cognate cellular receptor. Sci Rep 7, 42989, doi:10.1038/srep42989 (2017).
45 Arnold, J. N., Wormald, M. R., Sim, R. B., Rudd, P. M. & Dwek, R. A. The impact of glycosylation on the biological function and structure of human immunoglobulins. Annu Rev Immunol 25, 21-50, doi:10.1146/annurev.immunol.25.022106.141702 (2007).
46 Gonzalez-Quintela, A. et al. Serum levels of immunoglobulins (IgG, IgA, IgM) in a general adult population and their relationship with alcohol consumption, smoking and common metabolic abnormalities. Clin Exp Immunol 151, 42-50, doi:10.1111/j.1365-2249.2007.03545.x (2008).
47 Arnold, J. N. et al. Human serum IgM glycosylation: identification of glycoforms that can bind to mannan-binding lectin. J Biol Chem 280, 29080-29087, doi:10.1074/jbc.M504528200 (2005).
48 Hennicke, J. et al. Glycan profile of CHO derived IgM purified by highly efficient single step affinity chromatography. Anal Biochem 539, 162-166, doi:10.1016/j.ab.2017.10.020 (2017).
49 Lim, M. S. et al. Validation of Rapi-Fluor method for glycan profiling and application to commercial antibody drugs. Talanta 198, 105-110, doi:10.1016/j.talanta.2019.01.093 (2019).
50 Rohrer, J. S., Basumallick, L. & Hurum, D. C. Profiling N-linked oligosaccharides from IgG by high-performance anion-exchange chromatography with pulsed amperometric detection. Glycobiology 26, 582-591, doi:10.1093/glycob/cww006 (2016).
51 Gandham, S. et al. Technologies and Standardization in Research on Extracellular Vesicles. Trends Biotechnol 38, 1066-1098, doi:10.1016/j.tibtech.2020.05.012 (2020).
52 Kreimer, S. et al. Mass-spectrometry-based molecular characterization of extracellular vesicles: lipidomics and proteomics. J Proteome Res 14, 2367-2384, doi:10.1021/pr501279t (2015).
53 Macedo-da-Silva, J., Santiago, V. F., Rosa-Fernandes, L., Marinho, C. R. F. & Palmisano, G. Protein glycosylation in extracellular vesicles: Structural characterization and biological functions. Mol Immunol 135, 226-246, doi:10.1016/j.molimm.2021.04.017 (2021).
54 Wheeler, S. F. & Harvey, D. J. Negative ion mass spectrometry of sialylated carbohydrates: discrimination of N-acetylneuraminic acid linkages by MALDI-TOF and ESI-TOF mass spectrometry. Anal Chem 72, 5027-5039, doi:10.1021/ac000436x (2000).
55 Sagi, D., Peter-Katalinic, J., Conradt, H. S. & Nimtz, M. Sequencing of tri- and tetraantennary N-glycans containing sialic acid by negative mode ESI QTOF tandem MS. J Am Soc Mass Spectrom 13, 1138-1148, doi:10.1016/S1044-0305(02)00412-9 (2002).
56 Harvey, D. J. &Abrahams, J. L. Fragmentation and ion mobility properties of negative ions from N-linked carbohydrates: Part 7. Reduced glycans. Rapid Commun Mass Spectrom 30, 627-634, doi:10.1002/rcm.7467 (2016).
57 Wang, Y L. et al. Glycosylation of Siglec15 promotes immunoescape and tumor growth. Am J Cancer Res 11, 2291-2302 (2021).
58 Cheray, M. et al. Glycosylation-related gene expression is linked to differentiation status in glioblastomas undifferentiated cells. Cancer Lett 312, 24-32, doi:10.1016/j.canlet.2011.07.027 (2011).
59 Martinez-Maza, R. et al. The role of N-glycosylation in transport to the plasma membrane and sorting of the neuronal glycine transporter GLYT2. J Biol Chem 276, 2168-2173, doi:10.1074/jbc.M006774200 (2001).
60 Wielgat, P., Holownia, A. & Braszko, J. J. Lipopolysaccharide changes sialylation pattern in the mouse central nervous system. J Physiol Pharmacol 63, 555-561 (2012).
61 Zhao, Y., Mahajan, G., Kothapalli, C. R. & Sun, X. L. Sialylation status and mechanical properties of THP-1 macrophages upon LPS stimulation. Biochem Biophys Res Commun 518, 573-578, doi:10.1016/j.bbrc.2019.08.089 (2019).
62 Rebelo, A. L. et al. Complete spatial characterisation of N-glycosylation upon striatal neuroinflammation in the rodent brain. J Neuroinflammation 18, 116, doi:10.1186/s12974-021-02163-6 (2021).
63 Agard, N. J. & Bertozzi, C. R. Chemical approaches to perturb, profile, and perceive glycans. Acc Chem Res 42, 788-797, doi:10.1021/ar800267j (2009).
64 Prescher, J. A. & Bertozzi, C. R. Chemical technologies for probing glycans. Cell 126, 851-854, doi:10.1016/j.cell.2006.08.017 (2006).
65 Prescher, J. A., Dube, D. H. & Bertozzi, C. R. Chemical remodelling of cell surfaces in living animals. Nature 430, 873-877, doi:10.1038/nature02791 (2004).

1. A method of glycan analysis, the method comprising the steps of:
(a) providing an open tube capillary electrophoresis instrument whose output provides an electrospray input for a mass spectrometer, a glycan release agent solution disposed within a capillary tube of the open tube capillary electrophoresis instrument, and a sample comprising a glycoprotein in an aqueous medium;
(b) introducing the sample into an inlet of a capillary tube of the open tube capillary electrophoresis instrument, whereby the glycoprotein contacts the glycan release agent solution;
(c) allowing the glycan release agent to release one or more glycan moieties from the glycoprotein without modification of glycan structure or composition;
(d) separating the released glycan moieties within the capillary tube using the open tube capillary electrophoresis instrument based on charge and hydrodynamic volume of the released glycan moieties to form a plurality of separated glycan moieties within the capillary tube;
(e) injecting the separated glycan moieties from an outlet of the capillary tube into the mass spectrometer, whereby the separated glycan moieties are ionized and fragmented to form a plurality of charged glycan fragments;
(f) separating and detecting the charged glycan fragments based on mass-to-charge ratio using the mass spectrometer; and
(g) analyzing the separated and detected charged glycan fragments, whereby one or more structural characteristics of said plurality of glycan moieties are determined.

2. The method of claim 1, wherein the glycan release agent is PNGase F, and the released glycan moieties are N-glycans.

3. The method of claim 1, wherein the one or more glycans are released, separated, fragmented, and analyzed from glycoproteins of 1 to about 20 single cells present in the sample.

4. The method of claim 3, wherein one or more glycans are released, separated, fragmented, and analyzed from glycoproteins of a single cell.

5. The method of claim 1, wherein the analyzing of step (g) results in complete structure elucidation of at least one glycan moiety.

6. The method of claim 5, wherein the complete structure elucidation comprises identification of all monosaccharides of the glycan moiety and glycan derivative moieties and identification of glycosidic linkages of the glycan moiety.

7. The method of claim 5, wherein the complete structure elucidation identifies an intact native structure of the glycan moiety.

8. The method of claim 6, wherein the structural elucidation comprises the identification of glycan moieties containing from 1 to about 20 sialic acid residues.

9. The method of claim 6, wherein the structural elucidation comprises the identification of glycan moieties containing from 1 to about 10 fucose residues.

10. The method of claim 1, wherein the analyzing of step (g) comprises using output of peak areas and intensities from the mass spectrometer.

11. The method of claim 1 wherein, in step (d), isomers of glycan moieties are separated within the open tube capillary electrophoresis instrument in a single run.

12. The method of claim 1, wherein the sample comprises one or more single cells and the method preserves integrity of analyzed single cells.

13. The method of claim 1, wherein the method enables identification and quantification of intact and native glycan moieties.

14. The method of claim 1, wherein the method does not comprise labeling or derivatizing any glycan moiety.

15. The method of claim 1, wherein the method does not comprise use of any other endoglycosidase or exoglycosidase.

16. The method of claim 1, wherein glycans from at least 10, at least 20, at least 30, at least 40, or at least 50 different glycoproteins are released, separated, fragmented, and analyzed in a single run.

17. The method of claim 1, wherein the sample has a volume of less than 1 μL.

18. The method of claim 17, wherein the sample has a volume of less than 1 nL.

19. The method of claim 1, wherein the sample is obtained from blood, plasma, a bodily fluid, a biopsy sample, a cell suspension, a subcellular fraction, or a cell culture, or an extract or fraction of any of the foregoing.

20. The method of claim 19, wherein the blood, plasma, bodily fluid, biopsy sample, cell suspension, subcellular fraction, cell culture, or extract or fraction thereof was obtained from a source previously contacted with a chemical or biological therapeutic agent.

21. The method of claim 1, wherein the sample is cell-free.

22. The method of claim 1, wherein the sample is not subjected to any purification, homogenization, chromatography, centrifugation, or fractionation prior to use in the method.

23. The method of claim 1, wherein the sample is subjected to purification, homogenization, chromatography, centrifugation, or fractionation prior to use in the method.

24. The method of claim 1, wherein the separated glycan moieties are subjected to electrospray ionization when injected into the mass spectrometer in step (e).

25. The method of claim 1, wherein capillary electrophoresis/tandem mass spectrometry (CE/MS/MS) is used to perform steps (b) through (f).

26. The method of claim 1, wherein hydrodynamic pressure, electrospray, or electrokinetic injection is used to introduce the sample into the inlet of the capillary tube in step (b).

27. The method of claim 1, wherein the method is capable of full structure elucidation of a glycan present in a sample in amounts over a range of at least 4 orders of magnitude.

28. The method of claim 1, wherein the method is used to aid in performing spatial glycomic profiling of single cells or non-cellular sub-nanogram samples.

29. The method of claim 1, wherein the method is used to aid in performing spatial multiomic profiling of single cells or non-cellular sub-nanogram samples.

30. The method of claim 29, wherein the multiomic profiling comprises glycomic profiling and one or more of proteomic, genomic, transcriptomic, and metabolomic profiling.

31. The method of claim 1, wherein the method is used to aid in diagnosis and/or treatment of a disease or medical condition, and wherein the sample is obtained from a subject suspected of having the disease or medical condition.
